(19) United States
(12) Patent Application Publication
(10) Pub. No.: US 2010/0199391 A1
(43) Pub. Date: Aug. 5, 2010

(54) ANTI-MALARIA VACCINE COMPOSITIONS AND USES THEREOF

(75) Inventors: Stanislas Tomavo, Marquette Lez Lille (FR); Steven Graham Ball, Bourghelles (FR); Christophe D'Hulst, Watterlos (FR); David Dauvillee, Villeneuve D'Ascq (FR)

Correspondence Address: Pepper Hamilton LLP, 400 Berwyn Park, 899 Cassatt Road, Berwyn, PA, 19312-1183, US

(73) Assignees: CENTRE NATIONAL DE LA RECHERCHE SCIENTIFIQUE (CNRS, Paris (FR); UNIVERSITE DE SCIENCES ET TECHOLOGIES DE LILLE, Villeneuve d'Ascq (FR)

(21) Appl. No.: 12/523,812

(22) PCT Filed: Jan. 23, 2008
(86) PCT No.: PCT/EP2008/050758

§ 371 Date: Apr. 5, 2010

(30) Foreign Application Priority Data
Jan. 23, 2007 (FR) 0752837

(51) Int. Cl. A01H 5/00; C07K 14/195; C07H 21/00; C12N 15/74; C12N 5/10; C12N 1/13; A61K 38/16

(52) U.S. Cl. 800/301; 530/350; 536/23.7; 435/320.1; 435/419; 435/257.2; 514/12

(57) ABSTRACT

The present invention relates to a polypeptide in which: (i) the N-terminal sequence includes the polypeptide sequence of the GBSSI (Granule Bound Starch Synthase I) or a derivative or fragment thereof, wherein said polypeptide sequence can bind to starch; and (ii) the C-terminal sequence includes a polypeptide sequence coding for at least one epitope of an antigen from a parasite of the Plasmodium type; the invention also relates to a polynucleotide coding for such polypeptide, a vector containing said polynucleotide, a cell transformed by such vector, a transformed organism including such transformed cell, a pharmaceutical composition containing such polypeptide, and the use of such polypeptide in the preparation of a drug for the prophylactic treatment of a subject suffering from a pathology associated with a Plasmodium-type parasite, preferably malaria.

The present invention relates to malaria and, more specifically, to a polypeptide intended for anti-malaria vaccination, a pharmaceutical composition containing the same and the use of a such polypeptide for the preparation of a composition intended for the prophylactic treatment of malaria.

Malaria, which results from an infection by a parasite of the genus Plasmodium, today remains a major global endemic disease with nearly 300 to 500 million cases and more than 2.5 million deaths per year, primarily children younger than five (World Health Organisation Tropical Disease Research, TDR Twelfth Program Report, p. 57-76, 1997). Today, more than 40% of the global population lives in regions where malaria is rife.

Various prophylactic and therapeutic treatments have been developed against this parasite, the most effective molecules acting during erythrocyte infection phase. Examples include chloroquine (NIVAQUINE), halofantrine (HALFAN), mefloquine (LARIAM) and quinine; quinine remaining the reference drug to date. However, many of these molecules have significant side effects and it was noted the appearance of resistances developed by the more dangerous parasite (Plasmodium falciparum) to certain anti-malaria drugs in certain geographic regions.

There are today numerous approaches to research into an anti-malaria vaccine, and nearly 40 antigens usable in the development of a vaccine have been identified, said antigens being function of the stage of development of the parasite. For the intra-mosquito stage (sexual stages), one can distinguish: antigen Pfg27, Pfs16, Pfs25, Pfs28, Pfs45/48 or Pfs230; for the intravascular (sporozoite) stage: antigen CSP-1, STARP, SALSA or SSP-2; for the intrahepatic stage: antigen LSA-1, EXP-1, LSA-3, STARP, SALSA or SSP-2; and for the intra-erythrocyte (merozoite) stage: antigen RAP-1, RAP-2, SERA-1, MSP-1, MSP-2, MSP-3, MSP-4, MSP-5, AMA-1, EMP-1, Pf35, Pf55 or EBA-175.

As an example of an antigen, one can cite apical membranous antigen 1 (AMA1; accession number CAD42016 to CAD41967, CAD35671 to CAD35504, CAD34791 to CAD34741 and CAD31724 to CAD31720), which corresponds to a transmembranous protein identified originally in Plasmodium knowlesi (DEANS et al., Clin. Exp. Immunol., vol. 49, p: 297-309, 1982; DEANS et al., Mol. Biochem. Parasitol., vol. 11, p: 189-204; 1984) and, later, in other species of the genus Plasmodium (WATERS et al., J. Biol. Chem., vol. 265, p: 17974-17979, 1990). This protein is present in the apical organelles of the merozoite of the malaria parasite during the late schizont stage (HEALER et al., Infect. Immun., vol. 70, p: 5751-5758, 2002; BANNISTER et al., J. Cell. Sci., vol. 116, p: 3825-3834, 2003) or on the surface of the merozoite during the stage of schizont rupture and erythrocyte invasion (NARUM and THOMAS, Mol. Biochem. Parasitol., vol. 67, p:59-68, 1994). It has been shown that the presence of antibodies directed against antigen AMA1 prevents the invasion of erythrocytes (THOMAS et al., Mol. Biochem. Parasitol., vol. 13, p: 187-199, 1984; KOCKEN et al., J. Biol. Chem., vol. 273, p: 15119-15124, 1998; HODDER et al., Infect. Immun., vol. 69 (5), p: 3286-94, 2001), suggesting a critical role associated to this surface antigen. In addition, it has been shown that expression of the AMA1 protein is vital for the survival of the parasite (TRIGLIA et al., Mol. Microbiol., vol. 38, p: 706-718, 2000). Finally, the production of AMA1 in various systems has been described, but it proves to be problematic due to insufficient quantities (baculovirus) or to folding problems (E. coli) related to the large number of disulphide bridges.

One can also cite antigen MSP1 (Merozoite Surface Protein 1; accession number BAA2624 to BAA2604, AAQ20930 to AAQ20923, and AAC69750 to AAC69718), which is synthesised during the schizont stage and which is involved most notably in erythrocyte invasion by the parasite. During erythrocyte invasion, MSP1 undergoes several proteolytic cleavages during the merozoite maturation process. Among these cleavage products is a fragment corresponding to the C-terminal end of MSP1 and having a molecular weight of approximately 42 kDa (MSP1-42; HOLDER et al., Parasitology, vol. 94, p: 199-208, 1987; LYON et al., Proc. Natl. Acad. Sci. USA, vol. 83, p: 2989-2993, 1986). MSP1-42 remains attached to the membrane of the merozoite during the phase preceding the invasion. At the exact moment of invasion, MSP1-42 itself is divided into two fragments: an N-terminal fragment of approximately 33 kDa and a C-terminal fragment of approximately 19 kDa (MSP1-19; BLACKMAN et al., Mol. Biochem. Parasitol., vol. 50, p: 307-316, 1992). This last cleavage is essential for the success of the invasion, although the mechanism of the process remains unclear. Due to difficulties in producing protein MSP1 related to its large size (approximately 200 kDa), researchers have primarily studied the C-terminal portion which certainly presents the most important function (as yet unknown). Finally, the production of recombinant proteins comprising the C-terminal portion of MSP1 have been described with both a p19 and a p40 fused with a glutathione-S-transferase produced in E. coli (BURGHAUS et al., Infection and Immunity, vol. 64, p: 3614-3619, 1996; KUMAR et al., Molecular Medicine, vol. 1 (3), p: 325-332, 1995), or a p19 fused with a polypeptide derived from a tetanus toxoid and carrying helper T cell epitopes produced in S. cerevisiae (KUMAR et al., 1995, cited above). However, these various recombinant proteins have shown variable effectiveness in terms of antibody production after injection in monkeys.

Finally, and in spite of the significant efforts made by the international scientific community, the various vaccine candidates tested have shown only relative and very limited effectiveness for protection against malaria. In addition, the fact that malaria is found primarily in developing countries demands that a possible vaccine meets 3 essential criteria: (1) immediate effectiveness, (2) easy administration and (3) low cost.

It is in this context that the inventors developed a novel strategy consisting of the use of starch as a vector carrying epitopes of Plasmodium falciparum in order to produce a vaccine.

Starch, the archetypal plant kingdom's reserve polymer, represents one of the most important sources of polysaccharides present on Earth and can, most notably, be found in plants (maize, potato, wheat, rice, barley, etc.), algae, microalgae, etc. Starch occurs in the form of insoluble grains, whose size can vary from 0.1 micron to several tens of microns in diameter, and is composed of two polysaccharide sub-fractions called amylose and amylopectin which, respectively, account for approximately 25% and 75% by weight of the starch grain. Being composed only of glucose residues bound by alpha-1,4 bonds and branched at alpha-1,6, these two fractions differ both in structure as well as in the nature of the enzymatic functions involved in their synthesis. Whereas amylopectin (the major fraction of the starch grain responsible for its crystalline character) requires a complex set of enzymes for its production, the formation of amylose utilises only one particular starch synthetase called GBSS (granule bound starch synthase).

Several isoforms of GBSS have been isolated in maize, pea, potato or wheat (MACDONALD and PREISS, Plant Physiology, vol. 78, p: 849-852, 1985; SMITH, Planta, vol. 182, p: 599-604, 1990; DRY et al., The Plant Journal, vol. 2 (2), p: 193-202, 1992; DENYER et al., Planta, vol. 196, p: 256-265, 1995). In all cases, GBSSI represents the major isoform, said isoform being involved in amylose formation during biogenesis of the starch grain (TSAI, Biochemical Genetics, vol. 11 (2), p: 83-95, 1974; HOVENKAMP-HERMELINK et al., Theoretical and Applied Genetics, vol. 75, p: 217-221, 1987; DELRUE et al., Journal of Bacteriology, vol. 174 (11), p: 3612-3620, 1992; DENYER et al., 1995, cited above) and mutants defective for this enzyme containing only amylopectin.

The enzyme GBSSI uses ADP-glucose to bind together glucose residues via alpha-1,4 bonds, giving rise to long chains with little branching characteristic of the amylose fraction. A remarkable characteristic of this enzyme rests on the need to be bound to a polysaccharide matrix to carry out the enzymatic reaction. Thus, the enzyme is observed only when trapped within the starch grain and it represents nearly 1% of the dry weight of the starch under culture conditions optimal to its production. This abundance and the atypical localisation of the enzyme provide numerous technological advantages. GBSSI thus can be very easily purified using routine protocols, at laboratory as well as at industrial scale. The purified starch containing the protein can also be stored without notable degradation for months at room temperature. The particular localisation of the protein of interest (within the starch grain) also makes it possible to be free from the presence of plant allergens often observed during the preparation of recombinant proteins in plants.

A cDNA corresponding to the Waxy protein (by abuse of language, the term Waxy protein is used to designate GBSSI in plants, thus distinguish itif from the other GBBSGBSS) was isolated in wheat, barley, maize, rice, potato and pea. This approximately 60 kDa protein has a highly conserved sequence in the plant kingdom (AINSWORTH et al., Plant Mol. Biol., vol. 22 (1), p: 67-82, 1993). A remarkable exception should, however, be noted in the case of the unicellular green alga Chlamydomonas reinhardtii, where the enzyme has a non-relevant 12 kDa carboxy-terminus extension sequence certainly arising from a fusion with another gene (WATEFELD et al., Eur. J. Biochem., vol. 269 (15), p: 3810-20, 2002). A mutant strain of Chlamydomonas isolated by Steven Ball's team produces a 4 kDa truncated protein of this extension but maintains its ability to bind inside the starch grain.

The inventors fused a peptide carrying epitopes of Plasmodium falciparum with the GBSSI protein of the alga Chlamydomonas. They then showed that the fusion protein comprising the parasitic peptide MSP1 produced in the starch of the alga is highly immunogenic in rabbits and mice, leading to the production of polyclonal antibodies which are able to recognise very specifically the native MSP1 antigen as well as the parasites. Finally, these polyclonal antibodies are also able in vitro to completely inhibit the penetration of Plasmodium falciparum into erythrocytes. These results now open the way towards the expression of other Plasmodium antigens in the Chlamydomonas alga, in particular theoretically invariant epitopes belonging to the antigens of apical structures, which should allow the development of tests of protection against infection and disease in murine models.

In international application PCT WO 00/71734, the inventors described the principle of protein fusion between a truncated or non-truncated GBSSI protein and a polypeptide of interest. However, this document makes no case for the use of such fusion proteins for vaccinale purposes nor for the possibility of obtaining fusion proteins in which the polypeptide corresponding to the epitope shows a correct folding. In any case, this document neither describes nor suggests fusion proteins comprising epitopes of a parasite of the genus Plasmodium that cause a targeted immune response after injection.

Consequently, a first object of the invention relates to a polypeptide wherein:
(i) the N-terminal sequence comprises the polypeptide sequence of GBSSI (Granule Bound Starch Synthase I) or a derivative or fragment thereof, said polypeptide sequence being capable of binding to starch; and,
(ii) the C-terminal sequence comprises a polypeptide sequence coding for at least one epitope of an antigen of a parasite of the genus Plasmodium.

The sequence of GBSSI in various species of plants, algae or microalgae is known to those persons skilled in the art or can be determined by simple, routine experiments due to the conservation of its sequence.

GBSSI polypeptide sequence means the polypeptide sequence of the mature GBSSI, after cleavage of the signal peptide of the GBSSI precursor.

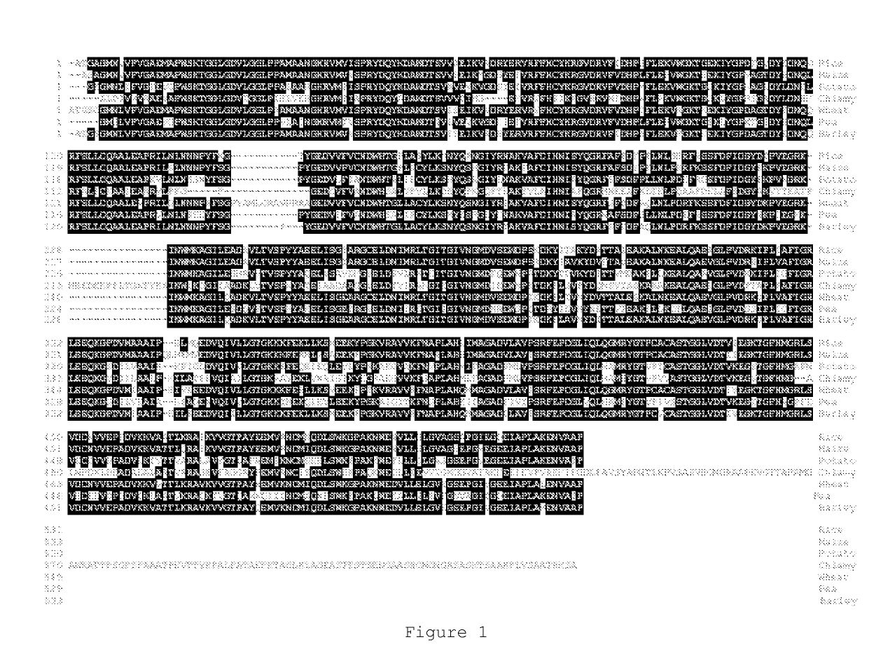

The sequence conservation of GBSSI protein between species is clearly illustrated in FIG. 1, which shows mature GBSSI sequence alignment in wheat (Triticum aestivum; accession number: P27736), Chlamydomonas reinhardtii (accession number: AAL28128), maize (Zea mays; accession number: P04713), pea (Pisum sativum; accession number: Q43092), rice (Oriza sativa; accession number: P19395), barley (Hordeum vulgare; accession number: P09842) and potato (Solanum tuberosum; accession number: Q00775).

Advantageously, the GBSSI polypeptide sequence is chosen from the group comprising GBSSI of Chlamydomonas reinhardtii (SEQ ID NO: 1), wheat (Triticum aestivum; SEQ ID NO: 2), maize (Zea mays; SEQ ID NO: 3), pea (Pisum sativum; SEQ ID NO: 4), rice (Oriza sativa; SEQ ID NO: 5), barley (Hordeum vulgare; SEQ ID NO: 6), potato (Solanum tuberosum; SEQ ID NO: 7), soya (Glycine max; SEQ ID NO: 8) and bean (Phaseolus vulgaris; SEQ ID NO: 9).

Preferably, the GBSSI polypeptide sequence corresponds to the GBSSI polypeptide sequence of Chlamydomonas reinhardtii (SEQ ID NO: 1), and in a particularly preferred manner to the polypeptide sequence from positions 1 to 527 of Chlamydomonas reinhardtii GBSSI (SEQ ID NO: 1).

Derived sequence means a sequence having a percentage of identity of at least 50%, for example of at least 65%, preferably of at least 80%, and in a particularly preferred manner of at least 95% with the GBSSI (Granule Bound Starch Synthase I) polypeptide sequence or a fragment thereof.

As an example, FIG. 1 shows that various GBSSI proteins of wheat, maize, pea, rice and potato share an identity greater than or equal to 50% with Chlamydomonas reinhardtii GBSSI.

Percentage of identity between two polypeptide sequences means the percentage of identical amino acids, between two sequences to be compared, obtained with the best possible alignment of said sequences. This percentage is purely statistical and the differences between the two sequences are distributed randomly over the entire length of the amino acid sequences. Better possible alignment or optimal alignment means alignment that produces the highest percentage of identity. Comparisons of sequences between two amino acid sequences are usually made by comparing said sequences after aligning them according to the best possible alignment; comparison is then made on comparison segments in order to identify and compare areas of similarity. The best possible alignment to make a comparison can be carried out by using the total homology algorithm of developed by SMITH and WATERMAN (Ad. App. Math., vol. 2, p: 482, 1981), by using the local homology algorithm developed by NEDDLEMAN and WUNSCH (J. Mol. Biol., vol. 48, p: 443, 1970), by using the similarity method developed by PEARSON and LIPMAN (Proc. Natl. Acd. Sci. USA, vol. 85, p: 2444, 1988), by using computer programs based on such algorithms (GAP, BESTFIT, BLAST P, BLAST N, FASTA, TFASTA, Genetics Group Computer, 575 Science Dr., Madison, Wis., USA), and by using MUSCLE multiple alignment algorithms (Edgar, Robert C., Nucleic Acids Research, vol. 32, p: 1792, 2004). To obtain the best possible alignment, preferably the BLAST program with the BLOSUM 62 matrix or the PAM 30 matrix will be used. The percentage of identity is established by comparing the two aligned sequences in an optimal fashion, said sequences possibly including additions or deletions in comparison with the reference sequence of a sort to obtain the best possible alignment between these two sequences. The percentage of identity is calculated by determining the number of identical positions between the two sequences, by dividing the number obtained by the total number of compared positions and by multiplying the result obtained by 100 to obtain the percentage of identity between these two sequences.

GBSSI fragment means a polypeptide of at least 50 amino acids, as an example of at least 100 or 150 amino acids, preferably of at least 200 amino acids, as an example of at least 250 or 350 amino acids, and in a particularly preferred manner a polypeptide of at least 400 amino acids.

As an example, the inventors have shown the fragment from position 1 to position 438 of the mature GBSSI in Chlamydomonas reinhardtii (SEQ ID NO: 1). Those persons skilled in the art are able to deduce, in view of their sole general knowledges, the corresponding GBSSI fragments in other species.

Preferably, the GBSSI fragments able to bind to starch are chosen from the group comprising the polypeptide sequence from positions 1 to 438 of Chlamydomonas reinhardtii GBSSI (SEQ ID NO: 1), 1 to 449 of wheat GBSSI (Triticum aestivum; SEQ ID NO: 2), 1 to 437 of maize GBSSI (Zea mays; SEQ ID NO: 3), 1 to 432 of pea GBSSI (Pisum sativum; SEQ ID NO: 4), 1 to 436 of rice GBSSI (Oriza sativa; SEQ ID NO: 5), 1 to 437 of barley GBSSI (Hordeum vulgare; SEQ ID NO: 6), 1 to 434 of potato GBSSI (Solanum tuberosum; SEQ ID NO: 7), 1 to 435 of soya GBSSI (Glycine max; SEQ ID NO: 8) and 1 to 431 of bean GBSSI (Phaseolus vulgaris; SEQ ID NO: 9).

Herein, antigen means a polypeptide able to trigger a humoral or cellular immune reaction.

Those persons skilled in the art, using their general knowledge, will be able to easily identify an antigen of a parasite of the genus Plasmodium, in particular of Plasmodium falciparum, Plasmodium vivax, Plasmodium ovale or Plasmodium malariae, preferably an antigen of Plasmodium falciparum or Plasmodium vivax, and a particularly preferred manner an antigen of Plasmodium falciparum.

Examples of such antigens include the following: Pfg27/25 (SEQ ID NO: 10), Pfs16 (SEQ ID NO: 11), Pfs25 (SEQ ID NO: 12), Pfs28 (SEQ ID NO: 13), Pfs48/45 (SEQ ID NO: 14), Pfs230 (SEQ ID NO: 15), CSP-1 (SEQ ID NO: 16), STARP (SEQ ID NO: 17), SALSA (SEQ ID NO: 18), SSP-2 (SEQ ID NO: 19), LSA-1 (SEQ ID NO: 20), EXP-1 (SEQ ID NO: 21), LSA-3 (SEQ ID NO: 22), RAP-1 (SEQ ID NO: 23), RAP-2 (SEQ ID NO: 24), SERA-1 (SEQ ID NO: 25), MSP-1 (SEQ ID NO: 26), MSP-2 (SEQ ID NO: 27), MSP-3 (SEQ ID NO: 28), MSP-4 (SEQ ID NO: 29), MSP-5 (SEQ ID NO: 30), AMA-1 (SEQ ID NO: 31), EMP-1 (SEQ ID NO: 32) and EBA-175 (SEQ ID NO: 33).

Epitope means a structure present on the surface of the antigen molecule able to bind to a single antibody molecule.

Advantageously, epitope means a polypeptide sequence derived from the polypeptide sequence of an antigen of a parasite of the genus Plasmodium.

Advantageously still, epitope means a polypeptide sequence of at least 6 amino acids, preferably of at least 8 amino acids, for example of at least 10 amino acids, and in a particularly preferred manner of at least 12 amino acids.

Those persons skilled in the art, using their general knowledge and simple, routine experiments, will be able to identify in the sequence of an antigen of a parasite of the genus Plasmodium, the polypeptide sequence coding for an epitope recognised specifically by an antibody. As an example, those skilled in the art will be able to use most notably the method described in the international application PCT WO02/30964.

Derived sequence means a polypeptide sequence presenting a percentage of identity of at least 70%, preferably of at least 80% and in a particularly preferred manner of at least 90% with the polypeptide sequence of an antigen of a parasite of the genus Plasmodium.

The polypeptide sequence of the epitope thus will be able to include substitutions (1, 2 or 3) with regard to the polypeptide sequence of an antigen of a parasite of the genus Plasmodium, of a sort that improve anchoring and thus the presentation of the polypeptide corresponding to said epitope by class II MHC molecules.

According to a preferred embodiment, the polypeptide sequence of said epitope presents 100% identity with the polypeptide sequence of an antigen of a parasite of the genus Plasmodium.

According to a preferred embodiment, said antigen corresponds to antigen MSP1 (SEQ ID NO: 26).

Preferably, the polypeptide sequence coding for at least one epitope of an antigen of a parasite of the genus Plasmodium is sequence SEQ ID NO: 34.

Advantageously, the inventive polypeptide has sequence SEQ ID NO: 35.

According to a second preferred embodiment, said antigen corresponds to antigen AMA-1 (SEQ ID NO: 31).

Preferably, the polypeptide sequence coding for at least one epitope of an antigen of a parasite of the genus Plasmodium is sequence SEQ ID NO: 36.

Advantageously, the inventive polypeptide has sequence SEQ ID NO: 37.

Advantageously, the inventive polypeptide comprises a linker polypeptide sequence between the N- and C-terminus sequences.

A second object of the invention relates to a polynucleotide coding for a polypeptide as described above.

Said polynucleotide corresponds to a DNA sequence, preferably said polynucleotide is a DNA sequence.

Advantageously, said polynucleotide codes for a polypeptide further presenting a signal peptide at its N-terminus, said signal peptide enabling intracellular transport of said polypeptide towards the sites of biosynthesis of starch grains. Preferably, said signal peptide corresponds to the GBSSI signal peptide of the organism in which the inventive polypeptide is produced.

As an example, the GBSSI signal peptide of Chlamydomonas reinhardtii (SEQ ID NO: 38) can be used for producing the polypeptide, according to the invention in this organism.

Advantageously still, the inventive polynucleotide is dependent operationally on a sequence of gene expression directing the expression of said polynucleotide in a eukaryotic cell, preferably in a plant, alga or microalga cell. Said sequence of gene expression corresponds to any regulatory sequence, such as a promoter sequence or a combination between a promoter sequence and an activator sequence facilitating the efficient transcription and translation of the polypeptide described above. Said gene expression sequence can correspond to a viral or eukaryotic promoter sequence, either constitutive or inducible. Examples of useful promoter sequences include higher-plant eukaryotic promoter sequences such as the promoters 35S and CaMV, or microalgae eukaryotic promoter sequences such as promoters HSP70, Rubisco, ARG7 (arginosuccinate lyase) and NIT1 (nitrate reductase).

A third object of the invention relates to a vector comprising the polynucleotide as described above.

Plasmids and phages can be cited as examples of such vectors.

A fourth object of the invention relates to a cell transformed by a vector as described above.

Preferably, said transformed cell is a plant cell containing one or more polynucleotides as described above, integrated in their genome or maintained in a stable manner in their cytoplasm, said plant cells being chosen among the cells of plants, algae or microalgae able to manufacture starch.

The transformation of plant cells can be carried out according to techniques well known to those persons skilled in the art. Examples of such techniques include electroporation, cytoplasmic or nuclear microinjection, gene gun or transformation using transformed bacteria such as Agrobacterium tumefaciens.

A fifth object of the invention further relates to a transformed organism such as a plant, alga or microalga that includes a transformed cell as described above.

Among the plants, algae or microalgae transformed in the context of the present invention include primarily wheat, maize, potato, rice, barley, amaranth, algae of the genus Chlamydomonas, in particular Chlamydomonas reinhardtii, and algae of the genus Chlorella, in particular Chlorella vulgaris.

A sixth object of the invention relates to a pharmaceutical composition including a polypeptide as described above, optionally combined with a pharmaceutically acceptable medium.

As examples of pharmaceutically acceptable media, the composition can include emulsions, microemulsions, oil in water emulsion, anhydrous lipids and water in oil emulsions, or other types of emulsions.

The inventive composition can further include one or more additives such as diluents, excipients, stabilisers and preservatives. Such additives are well known to those persons skilled in the art and are most notably described in Ullmann's Encyclopaedia of Industrial Chemistry, 6^{th} ed. (various editors, 1989-1998, Marcel Dekker); and in Pharmaceutical Dosage Forms and Drug Delivery Systems (ANSEL et al., 1994, WILLIAMS & WILKINS).

The polypeptide of the invention can be solubilised in a buffer, in water or can be incorporated in emulsions or microemulsions. As an example, usable buffers include phosphate buffered saline (PBS), physiological saline solution (150 mM NaCl in water) and Tris buffers.

There are many causes of instability or degradation of polypeptides such as hydrolysis and denaturation, which can lead to a decrease in the induction of the humoral or cellular response. Stabilisers can be added to decrease or prevent such problems.

Example of stabilisers include monosaccharides, disaccharides, polysaccharides, ionic and nonionic detergents, alkaline metal salts, phospholipids, fatty acids, polyols and stabilising peptides such as bovine serum albumin.

Advantageously, said at least one polypeptide is combined with starch grains.

The inventors have indeed shown that the production of the inventive polypeptide in combination with starch grains increases its stability.

The inventive composition can thus be provided in a form administrable by parenteral route, in particular by intravenous route, intraperitoneal route or in a form administrable by oral route.

Preferably, the above-mentioned pharmaceutical composition administrable by parenteral route is characterised in that the diameter of the starch grains is between 0.1 μm and several μm, in particular between approximately 0.1 μm and 10 μm. The proportion by weight of said at least one polypeptide in these starch grains is thus between 0.1% and 1% by weight.

Starch grains as described above whose small diameters are between 0.1 μm and 10 μm, and in which the proportion by weight of said at least one polypeptide as described above is between approximately 0.1% and 1%, are advantageously obtained:
from algae or microalgae transformed in the context of the present invention, mainly Chlamydomonas reinhardtii;
from plants or plant cells transformed in the context of the present invention and chosen for their ability to naturally produce the above-mentioned starch grains, said plants being in particular chosen among rice and amaranth;
from parts of plants transformed in the context of the present invention, said parts of these plants naturally producing the above-mentioned starch grains, such as plant leaves;
from plants or plant cells transformed in the context of the present invention, these plants being chosen among plants having mutations such that they produce the above-mentioned starch grains of small diameter, in particular from the mutant plants described in BULEON et al. (Int. J. Biol. Macromolecules, vol. 23, p: 85-112, 1998);
from plants or plant cells transformed in the context of the present invention, these plants being chosen among plants transformed using antisense nucleotide sequences for all or part of the gene coding for ADP-glucose pyrophosphorylase necessary to the synthesis of ADP-glucose in plant cells, in particular from the transformed plants described in MÜLLER-RÖBER et al. (EMBO J., vol. 11 (4), p: 1229-1238, 1992).

Advantageously, in the case of a pharmaceutical composition administrable via parenteral route, the starch grains are preferably chosen among those with amorphous structure if it is desired to rapidly release the polypeptide described above that they contain in the blood of the subject or, on the contrary, among those of crystalline structure when it is desired to gradually release said polypeptide in the blood.

As an illustration, amorphous starch grains can be obtained from seeds transformed according to the invention during the germination phase, or from particular mutant plants as described by SHANNON and GARWOOD (In starch: Chemistry and Technology, 2^{nd} edition, Academic Press, San Diego, Calif., p: 16-86, 1984), in particular from mutant cultivars such as the maize amylose-extender or all mutant cultivars of plants, algae or microalgae in which starch is amylose enriched.

As a further illustration, starch grains of crystalline structure advantageously have 30% to 35% crystals and can be obtained from plant seeds, in particular from cereals, having been just harvested and at maturity, or from mutant plants as described by SHANNON and GARWOOD (cited above, 1984), in particular from mutant cultivars such as maize waxy or all the mutant cultivars of plants, algae or microalgae in which the starch lacks amylose.

Such starch grains can be purified from plants, parts of plants, algae or microalgae using techniques well known to those persons skilled in the art. Examples of such techniques include the techniques described in HARRIS (The Chlamydomonas Sourcebook. A Comprehensive Guide to Biology and Laboratory Uses. Academic Press, San Diego, Calif., 1989) and in KINDLE (High-frequency nuclear transformation of Chlamydomonas reinhardtii. Genetics, 87, 1228-1232, 1990).

A seventh object of the invention relates to a method of prophylactic treatment of a pathology associated with a parasite of the genus Plasmodium comprising the administration of a therapeutically effective quantity of a composition as described above to a subject.

As used in the present application, the term “subject” refers to a mammal such as a rodent, feline, canine, primate or human, preferably said subject being human.

By pathology associated with a parasite of the genus Plasmodium is meant malaria in particular.

“Therapeutically effective quantity” means a quantity that induces the production of neutralising antibodies. Those persons skilled in the art will be able to determine said therapeutically effective quantity based on their general knowledge and/or simple, routine experiments.

An eighth object of the invention relates to the use of a composition as described above to manufacture a drug for the prophylactic treatment of a subject for a pathology associated with a parasite of the genus Plasmodium, preferably malaria.

According to a preferred embodiment, said composition is administered by parenteral route.

According to another preferred embodiment, said composition is administered by oral route.

The examples which follow are provided for illustrative purposes and in no way limit the scope of the present invention.

### EXAMPLES

### I Production of Fusion Proteins Including GBSSI and Antigens of a Parasite of the Genus Plasmodium

I-1 Obtaining Epitopes Derived from Antigens MSP1 and AMA-1 from Parasites of the Genus Plasmodium

In order to validate this concept of anti-malaria vaccination, two polypeptides from major antigens MSP1 and AMA-1 from parasites of the genus Plasmodium having proven their effectiveness in tests of protection against the parasite were selected (JOHN et al., J. Immunol., vol. 173, p: 666-72, 2004; MALKIN et al., Infect. Immun., vol. 73, p: 3677-85, 2005).

PCR using specific primers made it possible to amplify the polynucleotides coding for:
the 19 kDa C-terminus peptide (lacking the GPI glycolipid sequence) of the MSP1 (merozoite surface protein 1) major surface antigen of Plasmodium falciparum (human infection model); and,
the central region of AMA-1 (apical major antigen 1) of Plasmodium berghei (murine infection model).

More specifically, the 3′ primers used had an XhoI restriction site and enabled later phase-cloning of the polynucleotide obtained with the polynucleotide sequence coding for GBSSI. The 5′ primers had a BamHI restriction site.

The MSP1 and AMA-1 PCR fragments of 312 by and 336 bp, respectively, (SEQ ID NO: 39 and SEQ ID NO: 40) were purified and then cloned in the vector pCR2.1 using the TOPO TA Cloning Kit® (Invitrogen) according to the manufacturer's instructions to obtain the plasmids MSP1-pCR2.1 and AMA-1-pCR2.1, respectively.

I-2 Extraction of the Paro^{R} Resistance Gene of the pSG2 Plasmid of Chlamydomonas

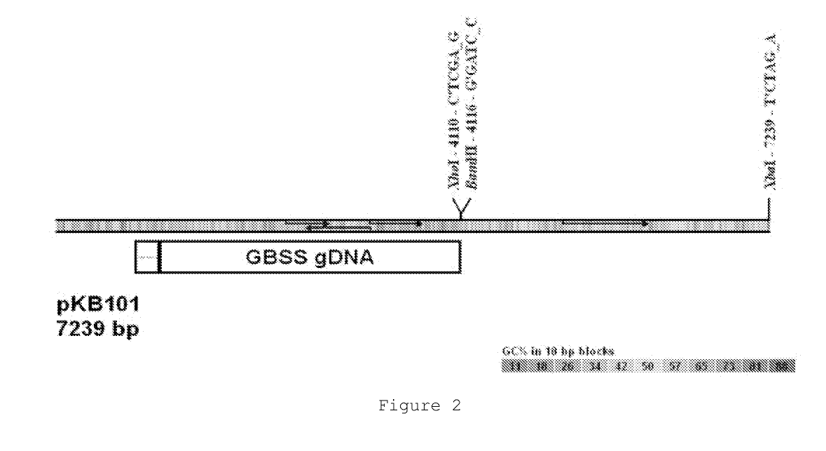

5 mg of plasmid pSG2 was digested for 2 hours at 37° C. with restriction enzyme XbaI. The 2300 by band of DNA which corresponds to the fragment conferring resistance to paromomycin (paro^{R}) was cut out and purified. The remaining plasmid fragment pSG2 (200 ng) was recircularized by T4 DNA ligase (BIOLABS) at 4° C., overnight, then one-fifth of the product was used to transform competent TOP10F′ bacteria (INVITROGEN) according to the manufacturer's instructions. In order to verify that the bacteria indeed included the pKB101 plasmid (see FIG. 2, which indicates the location of the troncated GBSSI and the cloning sites; SEQ ID NO: 41) and that the paro^{R} fragment was indeed removed, 4 clones was chosen and cultured in 5 ml of LB supplemented with ampicillin (50 μg/ml). Plasmid DNA was extracted using the NUCLEOSPIN PLASMID Kit (MACHEREY-NAGEL) according to the manufacturer's instructions, and then digested by the enzyme XbaI for 2 hours at 37° C. in order to verify the absence of the 2300 by paro^{R} fragment in the plasmid.

I-3 Extraction of Inserts MSP1 and AMA-1 of Plasmids MSP1-pCR2.1 and AMA-1-PCR2.1

The bacteria containing the plasmid MSP1-pCR2.1 were cultured in 50 ml of LB containing 50 μg/ml of ampicillin. The plasmid was then extracted with HISPEED PLASMID MIDI Kit® (QIAGEN) according to the manufacturer's instructions. 5 μg of plasmids MSP1-pCR2.1 was digested for 1 hour at 37° C. by the enzyme BamHI (BIOLABS), and then 1 hour at 37° C. by XhoI (BIOLABS) according to the manufacturer's instructions. The digestion product was then loaded on an agarose gel and 300 by band of DNA corresponding to the MSP1 fragment was purified, and then ligated with the enzyme T4 DNA ligase, as before, in the pSG2 plasmid digested beforehand with XhoI/BamHI to obtain the MSP1-pSG2′ plasmid. One-fifth of the ligated product was then used to transform competent TPO10F′ bacteria (INVITROGEN) as before.

In several positive clones resulting from this transformation, the presence of the plasmid MSP1-pSG2′ was confirmed by PCR and direct DNA sequencing.

The same protocol was used for plasmid AMA-1-pCR2.1 to obtain plasmid AMA1-pSG2′, which comprises a 312 by fragment corresponding to 104 amino acids of AMA1.

I-4 Insertion of the Paromomycin Resistance Gene (Paro^{R}) in Plasmids MSP1-pSG2′ and AMA1-pSG2′

Plasmid MSP1-pSG2′ (5 μg) was digested by the restriction enzyme XbaI for 2 hours at 37° C. Complete linearization of the plasmid was verified by loading an aliquot of the digestion on a 1% agarose gel.

The linearized MSP1-pSG2′ plasmid was then treated with alkaline phosphatase (ROCHE) and the paro^{R} fragment purified previously (cf. I-2) was inserted in the plasmid MSP1-pSG2′ to obtain plasmid MSP1-pSG2 using T4 DNA ligase (BIOLABS) according to the manufacturer's instructions. One-fifth of the ligation product was then used to transform competent TOP10F′ bacteria (INVITROGEN) and some positive clones were digested by the XbaI enzyme in order to verify the presence of a 2300 by insert.

The same procedure was used to construct plasmid AMA1-pSG2 from plasmid AMA1-pSG2′.

I-5 Transformation of the Alga Chlamydomonas with Plasmids MSP1-pSG2 and AMA1-pSG2

A mutant strain of Chlamydomonas, lacking endogenous GBSS, was transformed using the glass beads technique with 1 μg of plasmid MSP1-pSG2 according to the protocol described in KINDLE (1990, cited above).

Briefly, 300 μl of a cell suspension from a culture in TAP in exponential growth phase concentrated 100 times is vortexed vigorously in the presence of a microgram of DNA for 15 seconds. Six hundred microlitres of TAP are added and the cell suspension is spread on a dish (TAP supplemented with 10 μg/ml of paromomycin) to select the clones having integrated the MSP1-pSG2 plasmid. The paromomycin-resistant clones appear after 10 days at 23 degrees and under continuous light and are then screened in order to identify the clones expressing fusion protein GBSSI-MSP1 (SEQ ID NO: 35).

The same protocol was used with plasmid AMA1-pSG2 to obtain clones expressing fusion protein GBSSI-AMA-1 (SEQ ID NO: 37).

### II Expression of GBSSI-MSP1 and GBSSI-AMA-1 Proteins

Starch from various resistant clones likely to comprise fusion protein GBSSI-MSP1 was purified as described in HARRIS (1989, cited above).

Briefly, a culture of green alga Chlamydomonas reinhardtii in non-deficient medium (TAP) is centrifuged at 3000 g for 10 minutes at which point the cell density reaches 3 to 4 million cells per ml. The cell pellet is crushed using a French press (10,000 PSI). The lysate is then centrifuged (13,000 rpm, 15 minutes) and the pellet obtained containing starch and cellular debris is retained. This pellet is resuspended in 90% Percoll (GE HEALTHCARE) and then centrifuged (13,000 rpm, 45 minutes). The starch pellet obtained is washed twice with milliQ water (13,000 rpm, 10 minutes).

The starch assay is carried out using the kit sold by DIFFCHAMP (ENZYPLUS STARCH®) and then 10 mg aliquots are prepared. The starch can thus be preserved at 4 degrees for several months without apparent degradation. Proteins are then extracted from approximately 1 mg of starch for each clone with 60 μl of extraction buffer (5% β-mercaptoethanol (v/v), 2% SDS (w/v)) at 100° C. for 5 minutes. After centrifugation at 13,000 g for 10 minutes, the supernatant is recovered and the operation is repeated once with the pellet. The two supernatants are combined and then loaded on two SDS-PAGE electrophoresis gels. As a control, the same operations are carried out on starch from a strain of Chlamydomonas reinhardtii expressing wild GBSSI. One of the two gels is stained with Coomassie blue and the other is transferred to a nitrocellulose membrane (SCHLEICHER & SCHUELL) according to the manufacturer's instructions for Western blot analysis with a polyclonal antibody directed against the 19 kDa MSP1 parasitic peptide and then with a polyclonal antibody specific for Chlamydomonas reinhardtii GBSSI.

The gel stained with Coomassie revealed the existence of a majority band of approximately 70 kDa for the transformed mutant algae and of approximately 70 kDa for the wild alga, which corresponds to the size expected for GBSSI-MSP1 and GBSSI proteins, respectively. Indeed, the GBSS C-terminus peptide was removed and replaced by peptide MSP1, which makes it possible to have a recombinant protein of similar size. The difference between the transformants expressing GBSSI-MSP1 and the resistant strains not expressing it can be observed by the presence of a 70 kDa protein extracted from starch grains. Moreover, the results showed that said proteins are well recognised by the polyclonal antibody directed against GBSSI. On the other hand, only the majority protein expressed in the mutant strains and transformed by the MSP1-pSG2 vector is recognised by the polyclonal antibody directed against the 19 kDa MSP1 parasitic peptide.

The results further showed that fusion protein GBSS-AMA1 was also expressed successfully in starch of algae transformed by vector AMA1-pSG2.

### III Production of Polyclonal Antibodies Directed Specifically Against Fusion Protein GBSS-MSP1-19:

Ten milligrams of starch, containing fusion protein GBSS-MSP1 and purified as described above, was used to immunize five Balb/c mice with complete Freund's adjuvant followed by two boosters with incomplete adjuvant at one month intervals. Two weeks after the last immunization, serums from the five mice were collected.

In parallel, and as a negative control, ten milligrams of starch containing wild GBSS and purified as before was used to immunize five Balb/c mice according to the protocol described above.

The existence of antibodies directed against fusion protein GBSS-MSP1 in the collected serum was confirmed by Western blot analysis whose results showed that the serums obtained from the mice immunized with GBSS-MSP1 recognise specifically and uniquely the 190 kDa MSP1 major surface antigen as well as its various proteolytic peptides, such as the 70 kDa and 42 kDa peptides, as well as the membrane fragment of the infecting merozoites, the 19 kDa peptide. As expected, the serums produced against GBSS alone recognise no parasitic antigen, which confirms the specificity of the recognition of parasitic antigens containing the 19 kDa peptide epitopes.

The specificity of the anti-GBSS-MSP1 polyclonal serum against the 19 kDa MSP1 peptide was also confirmed by the indirect immunofluorescence technique (IFA). Indeed, and as expected, the results showed that said serum specifically recognised the membrane and the surface of Plasmodium falciparum inside infected erythrocytes whereas the anti-GBSS serums only are completely negative.

Finally, the expression of a fusion protein in starch thus allows not only its simple purification but also to easily test its immunogenicity since by simple absorption, starch will be transformed in the organism into glucose and antigenic proteins released during this process will thus become accessible to the immune system.

### IV Inhibition of the Invasion of Erythrocytes by Plasmodium falciparum:

A synchronous culture of Plasmodium falciparum containing schizonts was used. 900 μl of erythrocytes at 50% hematocrit and 1% parasitemia containing only schizonts was resuspended in 20 ml of complete RPMI. Next, 200 μl of the suspension was distributed in each well of a 96-well culture plate and 10 μl, 15 μl or 20 μl of healthy serum or anti-GBSS-MSP1 was added in a series of wells containing the parasitic suspension. For each concentration, 4 wells were tested.

The results showed that the specific polyclonal antibodies of GBSS-MSP1 completely inhibited (100% inhibition) invasion of the erythrocytes by Plasmodium falciparum. On the other hand, the polyclonal antibodies directed against starch alone did not display any inhibition of invasion of the erythrocytes by Plasmodium falciparum.

Finally, these results show that starch containing GBSS-MSP1 can thus elicit a humoral response containing polyclonal antibodies which neutralise the penetration of Plasmodium falciparum inside erythrocytes.

1. A polypeptide wherein:
(i) the N-terminal sequence comprises the polypeptide sequence of GBSSI (Granule Bound Starch Synthase I, a derivative or fragment thereof, said polypeptide sequence being capable of binding to starch; and,
(ii) the C-terminal sequence comprises a polypeptide sequence coding for at least one epitope of an antigen of a parasite of the genus Plasmodium.

2. The polypeptide according to claim 1, wherein the GBSSI polypeptide sequence is chosen from the group comprising GBSSI of Chlamydomonas reinhardtii (SEQ ID NO: 1), wheat (Triticum aestivum; SEQ ID NO: 2), maize (Zea mays; SEQ ID NO: 3), pea (Pisum sativum; SEQ ID NO: 4), rice (Oriza sativa; SEQ ID NO: 5), barley (Hordeum vulgare; SEQ ID NO: 6), potato (Solanum tuberosum; SEQ ID NO: 7), soya (Glycine max; SEQ ID NO: 8) and bean (Phaseolus vulgaris; SEQ ID NO: 9).

3. The polypeptide according to claim 2, wherein the GBSSI polypeptide sequence corresponds to the GBSSI polypeptide sequence of Chlamydomonas reinhardtii (SEQ ID NO: 1).

4. The polypeptide according to claim 1, wherein the GBSSI fragments able to bind to starch are chosen from the group comprising the polypeptide sequence from positions 1 to 438 of Chlamydomonas reinhardtii GBSSI (SEQ ID NO: 1), 1 to 449 of wheat GBSSI (Triticum aestivum; SEQ ID NO: 2), 1 to 437 of maize GBSSI (Zea mays; SEQ ID NO: 3), 1 to 432 of pea GBSSI (Pisum sativum; SEQ ID NO: 4), 1 to 436 of rice GBSSI (Oriza sativa; SEQ ID NO: 5), 1 to 437 of barley GBSSI (Hordeum vulgare; SEQ ID NO: 6), 1 to 434 of potato GBSSI (Solanum tuberosum; SEQ ID NO: 7), 1 to 435 of soya GBSSI (Glycine max; SEQ ID NO: 8) and 1 to 431 of bean GBSSI (Phaseolus vulgaris; SEQ ID NO: 9).

5. The polypeptide according to claim 1, wherein the parasite of the genus Plasmodium is chosen from the group comprising Plasmodium falciparum, Plasmodium vivax, Plasmodium ovale and Plasmodium malariae.

6. The polypeptide according to claim 1, wherein said antigen is chosen from the group comprising antigens Pfg27/25 (SEQ ID NO: 10), Pfs16 (SEQ ID NO: 11), Pfs25 (SEQ ID NO: 12), Pfs28 (SEQ ID NO: 13), Pfs48/45 (SEQ ID NO: 14), Pfs230 (SEQ ID NO: 15), CSP-1 (SEQ ID NO: 16), STARP (SEQ ID NO: 17), SALSA (SEQ ID NO: 18), SSP-2 (SEQ ID NO: 19), LSA-1 (SEQ ID NO: 20), EXP-1 (SEQ ID NO: 21), LSA-3 (SEQ ID NO: 22), RAP-1 (SEQ ID NO: 23), RAP-2 (SEQ ID NO: 24), SERA-1 (SEQ ID NO: 25), MSP-1 (SEQ ID NO: 26), MSP-2 (SEQ ID NO: 27), MSP-3 (SEQ ID NO: 28), MSP-4 (SEQ ID NO: 29), MSP-5 (SEQ ID NO: 30), AMA-1 (SEQ ID NO: 31), EMP-1 (SEQ ID NO: 32) and EBA-175 (SEQ ID NO: 33).

7. The polypeptide according to claim 1, wherein said epitope has a polypeptide sequence of at least 6 amino acids, preferably of at least 8 amino acids, derived from the polypeptide sequence of an antigen of a parasite of the genus Plasmodium.

8. The polypeptide according to claim 7, wherein said epitope consists of a polypeptide sequence presenting a percentage of identity of at least 70%, preferably of at least 80%, with the polypeptide sequence of an antigen of a parasite of the genus Plasmodium.

9. The polypeptide according to claim 8, wherein the polypeptide sequence of the epitope comprises substitutions with regard to the polypeptide sequence of an antigen of a parasite of the genus Plasmodium, of a sort that improve anchoring and thus the presentation of the polypeptide corresponding to said epitope by class II MHC molecules.

10. The polypeptide according to claim 7, wherein said epitope consists of a polypeptide sequence presenting 100% identity with the polypeptide sequence of an antigen of a parasite of the genus Plasmodium.

11. The polypeptide according to claim 6, wherein said antigen corresponds to antigen MSP1 (SEQ ID NO: 26).

12. The polypeptide according to claim 11, wherein the polypeptide sequence coding for at least one epitope of an antigen of a parasite of the genus Plasmodium is sequence SEQ ID NO: 34.

13. The polypeptide according to claim 12, wherein said polypeptide has sequence SEQ ID NO: 35.

14. The polypeptide according to claim 6, wherein said antigen corresponds to antigen AMA-1 (SEQ ID NO: 31).

15. The polypeptide according to claim 14, wherein the polypeptide sequence coding for at least one epitope of an antigen of a parasite of the genus Plasmodium is sequence SEQ ID NO: 36.

16. The polypeptide according to claim 15, wherein said polypeptide has sequence SEQ ID NO: 37.

17. The polypeptide according to claim 1, wherein it comprises a linker polypeptide sequence between the N- and C-terminus sequences.

18. The polypeptide according to claim 17, wherein said linker sequence comprises a peptide cleavage site making it possible to release the epitope after purification of said polypeptide.

19. A polynucleotide coding for a polypeptide according to claim 1.

20. A vector including a polynucleotide according to claim 19.

21. A cell transformed by a vector according to claim 20.

22. A transformed organism, wherein it comprises a cell according to claim 21.

23. A pharmaceutical composition including at least one polypeptide according to claim 1, optionally combined with a pharmaceutically acceptable medium.

24. The composition according to claim 23, wherein said at least one polypeptide is combined with starch grains.

25. Method for the prophylactic treatment of a subject for pathology associated with a parasite of the genus Plasmodium, preferably malaria, comprising the administration of an effective amount of a polypeptide according to claim 1 to a patient in need thereof.


SEQUENCE LISTING (41 sequences)

SEQ ID NO: 1 - protein, 624 aa, Chlamydomonas reihnarditii
ALDIVMVAAEVAPWSKTGGLGDVTGGLPIELVKRGHRVMTIAPRYDQYADAWDTSVVVDIMGEKVRYFHSIKKGVHRVWIDHPWFLAKVWGKTGSKLYGPRSGADYLDNHKRFALFCKAAIEAARVLPFGPGEDCVFVANDWHSALVPVLLKDEYQPKGQFTKAKSVLAIHNIAFQGRMWEEAFKDTKLPQAAFDKLAFSDGYAKVYTEATPMEEDEKPPLTGKTYKKINWLKGGIIAADKLVTVSPNYATEIAADAAGGVELDTVIRAKGIEGIVNGMDIEEWNPKTDKFLSVPYDQNSVYAGKAAAKEALQAELGLPVDPTAPLFAFIGRLEEQKGVDIILAALPKILATPKVQIAILGTGKAAYEKLVNAIGTKYKGRAKGVVKFSAPLAHMLTAGADFMLVPSRFEPCGLIQLHAMHYGTVPVVASTGGLVDTVKEGVTGFHMGALNPDKLDEADADALAATVRRASEVFAGGRYPEMVANCISQDLSWSKPAQKWEGLLEEVVYGKGGVATAKKEEIKVPVAEKIPGDLPAVSYAPNTLKPVSASVEGNGAAAPKVGTTAPAMGAWRATTPSGPSPAAATPKVTTYKPALPATAKPKTAGLKLAGEASTTSTSENGAAS

SEQ ID NO: 2 - protein, 544 aa, Triticum aestivum
ATGSGGMNLVFVGAEMAPWSKTGGLGDVLGGLPAAMAANGHRVMVISPRYDQYKDAWDTSVISEIKVVDRYERVRYFHCYKRGVDRVFVDHPCFLEKVRGTKEKIYGPDAGTDYEDNQQRFSLLCQAALEVPRILDLNNNPHFSGPYAMLCRAVPRRAGEDVVFVCNDWHTGLLACYLKSNYQSNGIYRTAKVAFCIHNISYQGRFSFDDFAQLNLPDRFKSSFDFIDGYDKPVEGRKINWMKAGILQADKVLTVSPYYAEELISGEARGCELDNIMRLTGITGIVNGMDVSEWDPIKDKFLTVNYDVTTALEGKALNKEALQAEVGLPVDRKVPLVAFIGRLEEQKGPDVMIAAIPEIVKEEDVQIVLLGTGKKKFERLLKSVEEKFPTKVRAVVRFNAPLAHQMMAGADVLAVTSRFEPCGLIQLQGMRYGTPCACASTGGLVDTIVEGKTGFHMGRLSVDCNVVEPADVKKVVTTLKRAVKVVGTPAYHEMVKNCMIQDLSWKGPAKNWEDVLLELGVEGSEPGIVGEEIAPLALENVAAP

SEQ ID NO: 3 - protein, 533 aa, Zea Mays
ASAGMNVVFVGAEMAPWSKTGGLGDVLGGLPPAMAANGHRVMVVSPRYDQYKDAWDTSVVSEIKMGDGYETVRFFHCYKRGVDRVFVDHPLFLERVWGKTEEKIYGPVAGTDYRDNQLRFSLLCQAALEAPRILSLNNNPYFSGPYGEDVVFVCNDWHTGPLSCYLKSNYQSHGIYRDAKTAFCIHNISYQGRFAFSDYPELNLPERFKSSFDFIDGYEKPVEGRKINWMKAGILEADRVLTVSPYYAEELISGIARGCELDNIMRLTGITGIVNGMDVSEWDPSRDKYIAVKYDVSTAVEAKALNKEALQAEVGLPVDRNIPLVAFIGRLEEQKGPDVMAAAIPQLMEMVEDVQIVLLGTGKKKFERMLMSAEEKFPGKVRAVVKFNAALAHHIMAGADVLAVTSRFEPCGLIQLQGMRYGTPCACASTGGLVDTIIEGKTGFHMGRLSVDCNVVEPADVKKVATTLQRAIKVVGTPAYEEMVRNCMIQDLSWKGPAKNWENVLLSLGVAGGEPGVEGEEIAPLAKENVAAP

SEQ ID NO: 4 - protein, 528 aa, Pisum savitum
GMSLVFVGAEVGPWSKTGGLGDVLGGLPPVLAGNGHRVMTVSPRYDQYKDAWDTNVLVEVKVGDKIETVRFFHCYKRGVDRVFVDHPLFLERVWGKTGSKLYGPKTGIDYRDNQLRFSLLCQAALEAPRVLNLNSSKYFSGPYGEDVIFVANDWHSALIPCYLKSMYKSRGLYKNAKVAFCIHNIAYQGRNAFSDFSLLNLPDEFRSSFDFIDGYNKPCEGKKINWMKAGILESDQVFTVSPHYAKELISGEDRGVELDNIIRSTGIIGIVNGMDNREWSPQTDRYIDVHYNETTVTEAKPLLKGTLQAEIGLPVDSSIPLIGFIGRLEEQKGSDILVEAIAKFADENVQIVVLGTGKKIMEKQIEVLEEKYPGKAIGITKFNSPLAHKIIAGADFIVIPSRFEPCGLVQLHAMPYGTVPIVSSTGGLVDTVKEGYTGFHAGPFDVECEDVDPDDVDKLAATVKRALKTYGTQAMKQIILNCMAQNFSWKKPAKLWEKALLNLEVTGNVAGIDGDEIAPLAKENVATP

SEQ ID NO: 5 - protein, 532 aa, Oriza sativa
ATGAGMNVVFVGAEMAPWSKTGGLGDVLGGLPPAMAANGHRVMVISPRYDQYKDAWDTSVVAEIKVADRYERVRFFHCYKRGVDRVFIDHPSFLEKVWGKTGEKIYGPDTGVDYKDNQMRFSLLCQAALEAPRILNLNNNPYFKGTYGEDVVFVCNDWHTGPLASYLKNNYQPNGIYRNAKVAFCIHNISYQGRFAFEDYPELNLSERFRSSFDFIDGYDTPVEGRKINWMKAGILEADRVLTVSPYYAEELISGIARGCELDNIMRLTGITGIVNGMDVSEWDPSKDKYITAKYDATTAIEAKALNKEALQAEAGLPVDRKIPLIAFIGRLEEQKGPDVMAAAIPELMQEDVQIVLLGTGKKKFEKLLKSMEEKYPGKVRAVVKFNAPLAHLIMAGADVLAVPSRFEPCGLIQLQGMRYGTPCACASTGGLVDTVIEGKTGFHMGRLSVDCKVVEPSDVKKVAATLKRAIKVVGTPAYEEMVRNCMNQDLSWKGPAKNWENVLLGLGVAGSAPGIEGDEIAPLAKENVAAP

SEQ ID NO: 6 - protein, 533 aa, Hordeum vulgare
ATGSGMNLVFVGAEMAPWSKTGGLGDVLGGLPPAMAANGHRVMVVSPRYDQYKDAWDTSVISEIKVADEYERVRFFHCYKRGVDRVFIDHPWFLEKVRGKTKEKIYGPDAGTDYEDNQQRFSLLCQAALEAPRILNLNNNPYFSGPYGEDVVFVCNDWHTGLLACYLKSNYQSNGIYRTAKVAFCIHNISYQGRFSFDDFAQLNLPDRFKSSFDFIDGYDKPVEGRKINWMKAGILQADKVLTVSPYYAEELISGEARGCELDNIMRLTGITGIVNGMDVSEWDPTKDKFLAVNYDITTALEAKALNKEALQAEVGLPVDRKVPLVAFIGRLEEQKGPDVMIAAIPEILKEEDVQIILLGTGKKKFEKLLKSMEEKFPGKVRAVVRFNAPLAHQMMAGADLLAVTSRFEPCGLIQLQGMRYGTPCVCASTGGLVDTIVEGKTGFHMGRLSVDCNVVEPADVKKVATTLKRAVKVVGTPAYQEMVKNCMIQDLSWKGPAKNWEDVLLELGVEGSEPGIVGEEIAPLAMENVAAP

SEQ ID NO: 7 - protein, 530 aa, Solanum tuberosum
GKGMNLIFVGTEVGPWSKTGGLGDVLGGLPPALAARGHRVMTISPRYDQYKDAWDTSVAVEVKVGDSIEIVRFFHCYKRGVDRVFVDHPMFLEKVWGKTGSKIYGPKAGLDYLDNELRFSLLCQAALEAPKVLNLNSSNYFSGPYGEDVLFIANDWHTALIPCYLKSMYQSRGIYLNAKVAFCIHNIAYQGRFSFSDFPLLNLPDEFRGSFDFIDGYEKPVKGRKINWMKAGILESHRVVTVSPYYAQELVSAVDKGVELDSVLRKTCITGIVNGMDTQEWNPATDKYTDVKYDITTVMDAKPLLKEALQAAVGLPVDKKIPLIGFIGRLEEQKGSDILVAAIHKFIGLDVQIVVLGTGKKEFEQEIEQLEVLYPNKAKGVAKFNVPLAHMITAGADFMLVPSRFEPCGLIQLHAMRYGTVPICASTGGLVDTVKEGYTGFHMGAFNVECDVVDPADVLKIVTTVARALAVYGTLAFAEMIKNCMSEELSWKEPAKKWETLLLGLGASGSEPGVEGEEIAPLAKENVATP

SEQ ID NO: 8 - protein, 531 aa, Glycine Max
MTFIIIGTEVAPWCKTGGLGDVLGGLPPALAGFGHRVMTIVPRYDQYKDAWDTSVVIEVKVGDRTEKVRFFHCYKRGVDRVFVDHPWFLEKVWGKTGQKLYGPTTGNDYEDNQLRFSLFCQAALEAPRVLSLNSSKYFSGPYGEDVIFVANDWHTALIPCYLKSMYQSRGIYTNARVVFCIHNIAYQGRFAFADFSLLNLPDQFKSSFDFIDGHVKPVVGRKINWLKAGLIESWFVITVSPNYAKELVSGPDKGVELDNIIRKIDDDGRLVGIVNGMDVQEWNPTTDKYIAVKYDVSTVLEAKALLKEALQAEVGLPVDRNIPLIGFIGRLEEQKGSDILAEAIPQFIKQNVQLVALGTGKKQMEKQLEELEISYPDKARGVAKFNVPLAHMIIAGADFILVPSRFEPCGLIQLQAMRYGSVPIVASTGGLVDTVKEGFTGFQMGAFNVECDAVDPADVDAISKTVKRALAVYGTPAFTEIIKNCMAQDLSWKGPAKEWEEVLLSLGVPGSEPGSDGEEIAPQAKENVATP

SEQ ID NO: 9 - protein, 527 aa, Phaseolus Vulgaris
MNLIFVGAEVAPWSKTGGLGDVLGGLPSALAEHGHRVMTVSPRYDQYKDAWDTNVTVEVKVADRIETVRFFHCYKQGVDRVFVDHPCFLEKVWGKTGSKLYGPSAGVDYEDNQLRYSLLCQAALEAPRVLNLNSNKYFSGPYGEDVIFVANDWHTALLPCYLKSMYQTRGVYRNTKVAFCIHNISYQGRHPFEDFPLLNLPNEYRSAFDFTDGHLKPVRGRKINWMKAAILESDLVLTVSPYYAKELVSGEDRGVELDNIIRKTGVAGIVNGMDIREWSPKTDKFIDIHFDTTSVKEAKFLLKEALQAEVGLPVNRDIPLIGFIGRLEEQKGSDILVEAIPKFIDQNVQIIILGTGKKSMEKQIEQLEEIYPEKARGIAKFDGPLAHKIIAGSDFIMIPSRFEPCGLVQLHSMPYGTVPIVSSTGGLVDTVQEGFTGFHMGAFNVDCEAIDPADVEKIATTVRRALGTYGTVAMEKIIQNCMAQDFSWKGPAKQWEKVLFSLDVGRSEAGIEGDEIAPLAKENVATP

SEQ ID NO: 10 - protein, 217 aa, Plasmodium falciparum
MSKVQKDSAKPLDKFGNIYDYHYEHETHAPLSPRIRKVGDIEFHACSDYIYLLMTLSKDPEKFNYALKDRVSIRRYVRKNQNRYNYFLIEERVQDNIVNRISDRLISYCTDKEVTEDYIKKIDDYLWVEQRVIEEVSINVDHAREVKEKKRIMNDKKLIRMLFDTYEYVKDVKFTDDQYKDAAARISQFLIDVVDSYIIKPIPALPVTPDEPHHNNI

SEQ ID NO: 11 - protein, 159 aa, Plasmodium Falciparum
MNIRKFIPSLALMLIFFAFANLVLSDANDKAKKPAGKGSPSTLQTPGSSSGASLHAVGPNLGGLSQGLSGKDSADKMPLETQLAIEEIKSLSNMLDKKTTVNRNLIISTAVTNMIMIKLIILSGIVGFKVKKTKNADDDKGDKDKDKDNTDEGDEGDDS

SEQ ID NO: 12 - protein, 217 aa, Plasmodium Falciparum
MNKLYSLFLFLFIQLSIKYNNAKVTVDTVCKRGFLIQMSGHLECKCENDLVLVNEETCEEKVLKCDEKTVNKPCGDFSKCIKIDGNPVSYACKCNLGYDMVNNVCIPNECKNVTCGNGKCILDTSNPVKTAVCSCNIGKVPNVQDQNKCSKDGETKCSLKCLKENETCKAVDGIYKCDCKDGFIIDNESSICTAFSAYNILNLSIMFILFSVCFFIM

SEQ ID NO: 13 - protein, 218 aa, Plasmodium Falciparum
MNTYFKVLLFLFIQLYITLNKARVTENTICKYGYLIQMSNHYECKCIEGYVLINEDTCGKKVVCDKVENSFKACDEYAYCFDLGNKNNEKQIKCMCRTEYTLTAGVCVPNVCRDKVCGKGKCIVDPANSLTHTCSCNIGTILNQNKLCDIQGDTPCSLKCAENEVCTLEGNYYTCKEDPSSNGGGNTVDQADTSYSVINGVTLTHVLIVCSIFIKLLI

SEQ ID NO: 14 - protein, 448 aa, Plasmodium Falciparum
MMLYISAKKAQVAFILYIVLVLRIISGNNDFCKPSSLNSEISGFIGYKCNFSNEGVHNLKPDMRERRSIFCTIHSYFIYDKIRLIIPKKSSSPEFKILPEKCFQKVYTDYENRVETDISELGLIEYEIEENDTNPNYNERTITISPFSPKDIEFFCFCDNTEKVISSIEGRSAMVHVRVLKYPHNILFTNLTNDLFTYLPKTYNESNFVSNVLEVELNDGELFVLACELINKKCFQEGKEKALYKSNKIIYHKNLTIFKAPFYVTSKDVNTECTCKFKNNNYKIVLKPKYEKKVIHGCNFSSNVSSKHTFTDSLDISLVDDSAHISCNVHLSEPKYNHLVGLNCPGDIIPDCFFQVYQPESEELEPSNIVYLDSQINIGDIEYYEDAEGDDKIKLFGIVGSIPKTTSFTCICKKDKKSAYMTVTIDSAYYGFLAKTFIFLIVAILLYI

SEQ ID NO: 15 - protein, 3135 aa, Plasmodium Falciparum
MKKIITLKNLFLIILVYIFSEKKDLRCNVIKGNNIKDDEDKRFHLFYYSHNLFKTPETKEKKNKKECFYKNGGIYNLSKEIRMRKDTSVKIKQRTCPFHKEGSSFEMGSKNITCFYPIVGKKERKTLDTIIIKKNVTNDHVVSSDMHSNVQEKNMILIRNIDKENKNDIQNVEEKIQRDTYENKDYESDDTLIEWFDDNTNEENFLLTFLKRCLMKIFSSPKRKKTVVQKKHKSNFFINSSLKYIYMYLTPSDSFNLVRRNRNLDEEDMSPRDNFVIDDEEEEEEEEEEEEEEEEEEEEEEEEEYDDYVYEESGDETEEQLQEEHQEEVGAESSEESFNDEDEDSVEARDGDMIRVDEYYEDQDGDTYDSTIKNEDVDEEVGEEVGEEVGEEVGEEVGEEVGEEVGEEVGEEVGEEEGEEVGEGVGEEVGEEEGEEVGEEEGEYVDEKERQGEIYPFGDEEEKDEGGESFTYEKSEVDKTDLFKFIEGGEGDDVYKVDGSKVLLDDDTISRVSKKHTARDGEYGEYGEAVEDGENVIKIIRSVLQSGALPSVGVDELDKIDLSYETTESGDTAVSEDSYDKYASNNTNKEYVCDFTDQLKPTESGPKVKKCEVKVNEPLIKVKIICPLKGSVEKLYDNIEYVPKKSPYVVLTKEETKLKEKLLSKLIYGLLISPTVNEKENNFKEGVIEFTLPPVVHKATVFYFICDNSKTEDDNKKGNRGIVEVYVEPYGNKINGCAFLDEDEEEEKYGNQIEEDEHNEKIKMKTFFTQNIYKKNNIYPCYMKLYSGDIGGILFPKNIKSTTCFEEMIPYNKEIKWNKENKSLGNLVNNSVVYNKEMNAKYFNVQYVHIPTSYKDTLNLFCSIILKEEESNLISTSYLVYVSINEELNFSLFDFYESFVPIKKTIQVAQKNVNNKEHDYTCDFTDKLDKTVPSTANGKKLFICRKHLKEFDTFTLKCNVNKTQYPNIEIFPKTLKDKKEVLKLDLDIQYQMFSKFFKFNTQNAKYLNLYPYYLIFPFNHIGKKELKNNPTYKNHKDVKYFEQSSVLSPLSSADSLGKLLNFLDTQETVCLTEKIRYLNLSINELGSDNNTFSVTFQVPPYIDIKEPFYFMFGCNNNKGEGNIGIVELLISKQEEKIKGCNFHESKLDYFNENISSDTHECTLHAYENDIIGFNCLETTHPNEVEVEVEDAEIYLQPENCFNNVYKGLNSVDITTILKNAQTYNINNKKTPTFLKIPPYNLLEDVEISCQCTIKQVVKKIKVIITKNDTVLLKREVQSESTLDDKIYKCEHENFINPRVNKTFDENVEYTCNIKIENFFNYIQIFCPAKDLGIYKNIQMYYDIVKPTRVPQFKKFNNEELHKLIPNSEMLHKTKEMLILYNEEKVDLLHFYVFLPIYIKDIYEFNIVCDNSKTMWKNQLGGKVIYHITVSKREQKVKGCSFDNEHAHMFSYNKTNVKNCIIDAKPKDLIGFVCPSGTLKLTNCFKDAIVHTNLTNINGILYLKNNLANFTYKHQFNYMEIPALMDNDISFKCICVDLKKKKYNVKSPLGPKVLRALYKKLNIKFDNYVTGTDQNKYLMTYMDLHLSHKRNYLKELFHDLGKKKPADTDANPESIIESLSINESNESGPFPTGDVDAEHLILEGYDTWESLYDEQLEEVIYNDIESLELKDIEQYVLQVNLKAPKLMMSAQIHNNRHVCDFSKNNLIVPESLKKKEELGGNPVNIHCYALLKPLDTLYVKCPTSKDNYEAAKVNISENDNEYELQVISLIEKRFHNFETLESKKPGNGDVVVHNGVVDTGPVLDNSTFEKYFKNIKIKPDKFFEKVINEYDDTEEEKDLESILPGAIVSPMKVLKKKDPFTSYAAFVVPPIVPKDLHFKVECNNTEYKDENQYISGYNGIIHIDISNSNRKINGCDFSTNNSSILTSSVKLVNGETKNCEININNNEVFGIICDNETNLDPEKCFHEIYSKDNKTVKKFREVIPNIDIFSLHNSNKKKVAYAKVPLDYINKLLFSCSCKTSHTNTIGTMKVTLNKDEKEEEDFKTAQGIKHNNVHLCNFFDNPELTFDNNKIVLCKIDAELFSEVIIQLPIFGTKNVEEGVQNEEYKKFSLKPSLVFDDNNNDIKVIGKEKNEVSISLALKGVYGNRIFTFDKNGKKGEGISFFIPPIKQDTDLKFIINETIDNSNIKQRGLIYIFVRKNVSENSFKLCDFTTGSTSLMELNSQVKEKKCTVKIKKGDIFGLKCPKGFAIFPQACFSNVLLEYYKSDYEDSEHINYYIHKDKKYNLKPKDVIELMDENFRELQNIQQYTGISNITDVLHFKNFNLGNLPLNFKNHYSTAYAKVPDTFNSIINFSCNCYNPEKHVYGTMQVESDNRNFDNIKKNENVIKNFLLPNIEKYALLLDDEERQKKIKQQQEEEQQEQILKDQDDRLSRHDDYNKNHTYILYDSNEHICDYEKNESLISTLPNDTKKIQKSICKINAKALDVVTIKCPHTKNFTPKDYFPNSSLITNDKKIVITFDKKNFVTYIDPTKKTFSLKDIYIQSFYGVSLDHLNQIKKIHEEWDDVHLFYPPHNVLHNVVLNNHIVNLSSALEGVLFMKSKVTGDETATKKNTTLPTDGVSSILIPPYVKEDITFHLFCGKSTTKKPNKKNTSLALIHIHISSNRNIIHGCDFLYLENQTNDAISNNNNNSYSIFTHNKNTENNLICDISLIPKTVIGIKCPNKKLNPQTCFDEVYYVKQEDVPSKTITADKYNTFSKDKIGNILKNAISINNPDEKDNTYTYLILPEKFEEELIDTKKVLACTCDNKYIIHMKIEKSTMDKIKIDEKKTIGKDICKYDVTTKVATCEIIDTIDSSVLKEHHTVHYSITLSRWDKLIIKYPTNEKTHFENFFVNPFNLKDKVLYNYNKPINIEHILPGAITTDIYDTRTKIKQYILRIPPYVHKDIHFSLEFNNSLSLTKQNQNIIYGNVAKIFIHINQGYKEIHGCDFTGKYSHLFTYSKKPLPNDDDICNVTIGNNTFSGFACLSHFELKPNNCFSSVYDYNEANKVKKLFDLSTKVELDHIKQNTSGYTLSYIIFNKESTKLKFSCTCSSNYSNYTIRITFDPNYIIPEPQSRAIIKYVDLQDKNFAKYLRKL

SEQ ID NO: 16 - protein, 403 aa, Plasmodium Falciparum
ALFQEYQCYGSSSNTRVLNELNYDNAGTNLYNELEMNYYGKQENWYSLKKNSRSLGENDDGNNNNGDNGREGKDEDKRDGNNEDNEKLRKPKHKKLKQPGGDNPDPNANPNVDPNANPNANPNANPNANPNANPNANPNANPNANPNANPNANPNANPNANPNANPNVDPNANPNANPNANPNANPNANPNANPNANPNANPNANPNANPNANPNANPNANPNANPNANPNANPNANPNANPNANPNANPNANPNANPNANPNANPNANPNANPNANPNANPNANPNANPNANPNKNNQGNGQGHNMPNDPNRNVDENANANNAVKNNNNEEPSDKHIEQYLKKIQNSLSTEWSPCSVTCGNGIQVRIKPGSANKPKDELDYENDIEKKICKMEKCSSVFNVV

SEQ ID NO: 17 - protein, 559 aa, Plasmodium Falciparum
MIHIFYKTAIFTLSIWTTLLYSNKNLKCNFYYNNNNLSTYVIKHNRFLSEYQSNFLGGGYSAALKLVNSKKSGTNVNTKYNSENTNTNNNIPESSSTYTNTRLPANNSTTTSTTKVTDNNKTNIKLTGNNSTTINTNSTENTSATKKVTENVITNQILTGNNNTTTNTSTTEHNNNINTNTNSTDNSNTNTNLTDNTSTTKKLTDNINTTQNLTTSTNTTTVSTDNNNINTKPIDNNNTDIKSTDNYNTGTKETDNKNTDIKATDNNNITTTTDNTNTNVISTDNSKTNVISTDNSKTNTISTDNDNADTILTDNDNNTDIILTDNNDTDTISTDNDNADTKATDNNNNTNTKATDNNNTKIISPDNNNTKTASTDNNNNTKIISPDNNNTKTISTDNNNTKAISTDNNNTKTISTDNNNTKTISNDNNNTNTISTDNNNNNTNQYVFANNYNETTSDDELNKDSCDYSEEKENIKSMINAYLDKLDLETVRKIHSDISTCIEKKNNPRNQITHLNNLKNMYNIIKFIVVIYIAFNWSEVIHKYVGKLILAFALYMLIN

SEQ ID NO: 18 - protein, 83 aa, Plasmodium Falciparum
RVSTSDTPGGNESSSAFPQFIWSAEKKDEKEASEQGEESHKKENSQESANGKDDVKEEKKTNEKKDDGKTDKVQEKVLEKSPK

SEQ ID NO: 19 - protein, 559 aa, Plasmodium Falciparum
MNHLGNVKYLVIVFLIFFDLFLVNGRDVQNNIVDEIKYREEVCNDEVDLYLLMDCSGSIRRHNWVNHAVPLAMKLIQQLNLNESAIHLYVNIFSNNAKEIIRLHSDASKNKEKALIIIRSLLSTNLPYGRTNLSDALLQVRKHLNDRINRENANQLVVILTDGIPDSIQDSLKESRKLNDRGVKIAVFGIGQGINVAFNRFLVGCHPSDGKCNLYADSAWENVKNVIGPFMKAVCVEVEKTASCGVWDEWSPCSVTCGKGTRSRKREILHEGCTSELQEQCEEERCPPKREPLDVPDEPEDDQPRPRGDNFAVEKPEENIIDNNPQEPSPNPEEGKGENPNGFDLDENPENPPNPDIPQQEPNIPEDSEKEVPSDVPKNPEDDREENFDIPKKPENKHDNQNNLPNDKSDRSIPYSPLPPKVLDNERKQSDPQSQDNNGNRHVPNSEDRETRPHGRNNENRSYNRKYNDTPKHPEREEHEKPDNNKKKGGSDNKYKIAGGIAGGLALLACAGLAYKFVVPGAATPYAGEPAPFDETLGEEDKDLDEPEQFRLPEENEWN

SEQ ID NO: 20 - protein, 493 aa, Plasmodium Falciparum
QEQQSDLEQERRAKEKLQEQQSDLEQDRLAKEKLQEQQSDLEQERLAKEKLQEQQSDLEQERRAKEKLQEQQSDLEQERRAKEKLQEQQSDLEQDRLAKEKLQEQQSDLEQERRAKEKLQEQQSDLEQERRAKEKLQEQQSDLEQERLAKEKLQEQQSDLEQERRAKEKLQEQQSDLEQERRAKEKLQEQQSDLEQERRAKEKLQEQQRDLEQRKADTKKNLERKKEHGDILAEDLYGRLEIPAIELPSENERGYYIPHQSSLPQDNRGNSRDSKEISIIEKTNRESITTNVEGRRDIHKGHLEEKKDGSIKPEQKEDKSADIQNHTLETVNISDVNDFQISKYEDEISAEYDDSLIDEEEDDEDLDEFKPIVQYDNFQDEENIGIYKELEDLIEKNENLDDLDEGIEKSSEELSEEKIKKGKKYEKTKDNNFKPNDKSLYDEHIKKYKNDKQVNKEKEKFIKSLFHIFDGDNEILQIVDELSEDITKYFMKL

SEQ ID NO: 21 - protein, 162 aa, Plasmodium Falciparum
MKILSVFFLVLFFIIFNKESLAEKTNKETGSGVSSKKKNKKGSGEPLIDVHDLISDMIKKEEELVEVNKRKSKYKLATSVLAGLLGVVSTVLLGGVGLVLYNTEKGRHPFKIGSSDPADNANPDADSESNGEPNADPQVTAQDVTPEQPQGDDNNLVSGPEH

SEQ ID NO: 22 - protein, 393 aa, Plasmodium Falciparum
VEEKVEESVEENDEESVEENVEENVEENDDGSVASSVEESIASSVDESIDSSIEENVAPTVEEIVAPTVEEIVAPSVVESVAPSVEESVEENVEESVAENVEESVAENVEESVAENVEESVAENVEESVAENVEEIVAPTVEEIVAPSVVESVAPSVEESVEENVEESVAENVEESVAENVEESVAENVEEIVAPSVEESVAPTVEEIVAPSVEESVAPSVEESVAPSVEESVAENVEEIVAPSVEEIVAPSVEESVAENVEEIVAPSVEEIVAPSVEEIVAPSVEEIVAPTVEEIVVPTVEESVAPSVEESVAPSVEESVAENVEESVAENVEEIVAPSVEEIVAPSVEEIVAPSVEEIVAPTVEEIVAPSVEEIVAPTVEESVAENVATNL

SEQ ID NO: 23 - protein, 782 aa, Plasmodium Falciparum
MSFYLGSLVIIFHVLFRNVADGINVNGDNNYGKTIINNDFNFDDYNYWTPINKKEFLNSYEDEFSSESFLENKSSVDDGNINLTDTSTSNKSSKKGHGRSRVRSASAAAILEEDDSKDDMEFKASPSVVKTSTPSGTQTSGLKSSSPSSTKSSSPSNVKSASPHGESNSSEESTTKSSKRSASVAGIVGADEEAPPAPKNTLTPLEELYPTNVNLFNYKYSLNNMEENINILKNEGDLVAQKEEFEYDENMEKAKQDKKKALEKIGKESDEAPFMFSENKFLENQVKERNVAGSFSRFFSKLNPFKKDEVIEKTEVSKKTFSGIGFNLTDKEAKVLGVGATYQEYPETMLYNCPNNSNLFDTIESLQGRIIDIKKRESMISTTFEQQKECLKNMGVLDLELNDTQCKFGTCIGSFGEHHLRLYEFENDLFKFHPNIDYLTLADGYKLQKNHIYELSHVNFCLLNPKTLEEFLKKKEIKDLMGGDDLIKYKENFDNFMSISITCHIESLIYDDIEASQDIAAVLKIAKSKLHVITSGLSYKARKLVYKIYSEIQKNPDELYEKLTWIYDNIYMIKRYYTAYALEGVCSYLEHDKSQMYTELHIYNKIVDSVRYYSSCFKNVIVYNAIISGIHEKIKHFLKLVPRHNFLLDYHFNSIFEKEIKPAKKYSTSHIYFDPTVASYAYYNLDRRTMVTIINDYFEAKKKELTVIVSRMKTDMLSIQNEESKIPNDKSANSKLATRLMKKFKAEIRDFFKEMRIQYAKLINIRYRSHLKKNYFAFKRLD

SEQ ID NO: 24 - protein, 398 aa, Plasmodium Falciparum
MGLKFYVLVFLILCLKNVVKGDKCETEFSKLYPESNSLTGLIYAHTAHVHKLSMWVYFIYNHFSSADELIKYLEKTNINTLENSDHTCFARAVTLYLFYYYLKDIKSMLSTDDYQSFFKNKFKDINPLFINDFILILNDKKFMENLDLYIMKESEREHLVIKKNPFLRVLNKASTTTHATYKYNRYFIVGSRVHTPYKDYFGDFNKYTEISVLNYVRDYNFLIYAGSRENYYNSDIAGPARSVNNVISKNKTLGLRKRSSSLALVGTNNNDPIFAYCEKDNKSEYYGTPDDLITSFFSIIKTKMLNSHKTFLRQFDYALFHKTYSIPNLKGFRFLKHLFQKKNLVNFVGMYENHVSTEINFLAEDFVELFDVTMDCYSRQYSNRAAENFKAIRELNVL

SEQ ID NO: 25 - protein, 995 aa, Plasmodium Falciparum
MKSYISLFFILRVIFNKNVIKCTGESQTGNTGGGQVGNTVGGQAGNTVGDQAGSTGGSPQGSTGASQPGSSEPSNPVSSGHSVSTVSVSQTSTSSEKQDTIQVKSALLKDYMGLKVTGPCNENFIMFLVPHIYIDVDTEDTNIELRTTLKETNNAISFESNSGSLEKKKYVKLPSNGTTGEQGSSTGTVRGDTEPISGSSSSSSSSSSSSSSSSSSSSSSPSSSSSSSSSSESLPANGPDSPTVKPPRNLQNICETGKSFKLVVYIKENTLIIKWKVYGETKDTTENNKVDVRKYLINEKETPFTNILIHAYKEHNGTNLIESKNYSLGSDIPEKCDTLASNCFLSGNFNIEKCFQCALLVEKENKNDVCYKYLSEDIVSKFKEIKAETEDDDEDDYTEYKLTESIDNILVKMFKTNENNDKSELIKLEEVDDSLKLELMNYCSLLKDVDTTGTLDNYGMGNEMDIFNNLKRLLIYHSEENINTLKNKFRNAAVCLKNVDDWIVNKRGLVLPELNYDLEYFNEHLYNDKNSPEDKDNKGKGVVHVDTTLEKEDTLSYDNSDNMFCNKEYCNRLKDENNCISNLQVEDQGNCDTSWIFASKYHLETIRCMKGYEPTKISALYVANCYKGEHKDRCDEGSSPMEFLQIIEDYGFLPAESNYPYNYVKVGEQCPKVEDHWMNLWDNGKILHNKNEPNSLDGKGYTAYESERFHDNMDAFVKIIKTEVMNKGSVIAYIKAENVMGYEFSGKKVQNLCGDDTADHAVNIVGYGNYVNSEGEKKSYWIVRNSWGPYWGDEGYFKVDMYGPTHCHFNFIHSVVIFNVDLPMNNKTTKKESKIYDYYLKASPEFYHNLYFKNFNVGKKNLFSEKEDNENNKKLGNNYIIFGQDTAGSGQSGKESNTALESAGTSNEVSERVHVYHILKHIKDGKIRMGMRKYIDTQDVNKKHSCTRSYAFNPENYEKCVNLCNVNWKTCEEKTSPGLCLSKLDTNNECYFCYV

SEQ ID NO: 26 - protein, 393 aa, Plasmodium Falciparum
AISVTMDNILSGFENEYDVIYLKPLAGVYRSLKKQIEKNIFTFNLNLNDILNSRLKKRKYFLDVLESDLMQFKHISSNEYIIEDSFKLLNSEQKNTLLKSYKYIKESVENDIKFAQEGISYYEKVLAKYKDDLESIKKVIKEEKEFPSSPPTTPPSPAKTDEQKKESKFLPFLTNIETLYNNLVNKIDDYLINLKAKINDCNVEKNEAHVKITKLSDLKAIDDKIDLFKNHNDFEAIKKLINDDTKKDMLGKLLSTGLVQNFPNTIISKLIEGKFQDMLNISQHQCVKKQCPENSGCFRHLDEREECKCLLNYKQEGDKCVENPNPTCNENNGGCDADAKCTEEDSGSNGKKITCECTKPDSYPFFDGIFCSSSNFLGISFLLILMLILYSFI

SEQ ID NO: 27 - protein, 108 aa, Plasmodium Falciparum
MSIRRSMANEGSSTNSVGANAPKADTIASGSQSSTNSASTSTTNNRESQTTTPTAADTPTATESNSPSPPIATTESNSPSPPITTTESNSPSPPITTTESSKFWQCTN

SEQ ID NO: 28 - protein, 379 aa, Plasmodium Falciparum
MKSFINITLSLFLLHLYIYINNVASKEIVKKYNLNLRNAILNNNSQIENEEKDIKYELNEQNDENVNTTIVANSMEFGEGFSADDQKDIEAYKKAKQASQDAEQAAKDAEQAAKDAEQASKDAEKLKESDESYTKAKEACTAASKAKKAFETASNAKKAAESALKTNADEKPSRINLFSRKTKEYAEQVEKDYERAKNAYQKANQAVLKAKEASSYDYILGWEFGGGVPEHKKEENMLSHLYVSSKDKENISKENDDVLDEKEEEAEETEEEELEEKNEEETESEISEDEEEEEEEEKEEENDKKKEQEKEQSNENNDQKKDMEAQNLISKNQNNNEKNVKEAAESIMKTLAGLIKGNNQIDSTLKDLVEELSKYFKNH

SEQ ID NO: 29 - protein, 271 aa, Plasmodium Falciparum
MWIVKFLIVVHFFIICTINFDKLYISYSYNIVPENGRMLNMRILGGEKPNVDGVSTSDTPGGNESSSASPNLSDAAEKKDEKEASEQEESHKKENSQESANGKDDVKEEKKTNEKKDDGKTDKVQEKVLEKSPKESQMVDDKKKTEAIPKKVVQPSSSNSGGHVGEEEDHNEGEGEHEEEEEHEEDDDVEDDDTYNKDDLEDEDLCKHNNGDCGDDKLCEYVGNRRVKCKCKEGYKLEGIECVELLSLASSSLNLIFNSFITIFVVILLIN

SEQ ID NO: 30 - protein, 272 aa, Plasmodium Falciparum
MNILCILSYIYFFVIFYSLNLNNKNENFLVVRRLMNDEKGEGGFTSKNKENGNNNRNNENELKEEGSLPTKMNEKNSNSSDKQPNDISHDESKSNSNNSQNIQKEPEEKENSNPNLDSSENSSESATRSVDISEHNSNNPETKEENGEEPLDLEINENAEIGQEPPNRLHFDNVDDEVPHYSALRYNKVEKNVTDEMLLYNMMSDQNRKSCAINNGGCSDDQICININNIGVKCICKDGYLLGTKCIILNSYSCHPFFSILIYITLFLLLFV

SEQ ID NO: 31 - protein, 556 aa, Plasmodium Falciparum
MKEIYYILILCSIYLINLSNCSEGPNNVISENGHINYDMIQKENTERSTKLINPWEKYTEKYDIERMHGSGIRVDLGEDARVENRDYRIPSGKCPVIGKGITIQNSEVSFLTPVATGDQSVRSGGLALPKTDVHLSPITIDNLKTMYKEHTEIVKLNNMSLCAKHTSFYVPGNNANSAYRHPAVYDKSNSTCYMLYVAAQENMGPRYCSNNANNDNQPFCFTPEKIEKYKNLSYLTKNLRDDWETSCPNKSIKNAKFGIWVDGYCKDYQKHTVHDSDSLLKCNQIIFNESASDQPKQYEKHLEDTTKFRQGVAERNGKLIGEALLPIGSYKSDQIKSHGRGYNWGNYDSQNKKCYIFETKPTCLINDRNFIATTALSSTEEFEEQFPCDIYKNKINEEIKVLNKNISNGNNSIEFPRIFISTDKNSLNCPCEPTQLTESSCNFYVCNCVEKRQYIAENNDVEIKEELEVHMKAHQTREVIVIIIFICVGIILVILLVGYFFKSNKKGENYDRMGQADIYGKANSRKDGMLDPEVSFWGEDKRASHTTPVLMEKPYY

SEQ ID NO: 32 - protein, 118 aa, Plasmodium Falciparum
ADIGDIVRGKDMFKPNDADKVEKGLQVVFKKIYEGFLDKGVKVHYKENKDGNYVKLREDWWMANRDQVWRAITCKAPGDVNYFRKISEDTRKFENAGKCRRHDNSVPTNLDYVPQFLR

SEQ ID NO: 33 - protein, 579 aa, Plasmodium Falciparum
GRNTSSNNEVLSNCREKRKGMKWDCKKKNDRSNYVCIPDRRIQLCIVNLSIIKTYTKETMKDHFIEASKKESQLLLKKNDNKYNSKFCNDLKNSFLDYGHLAMGNDMDFGGYSTKAENKIQEVFKGAHGKISEHEIKNFRKEWWNEFREKLWEAMLSEHKNNINNCKNIPQEELQITQWIKEWHGEFLLERDNRSKLPKSKCKNNTLYEACEKECIDPCMKYRDWIIRSKFEWHTLSKEYETQKVPKENAENYLIKISENKNDAKVSLLLNNCDAEYSKYCDCKHTTTLVKSVLNGNDNTIKEKREHIDLDDFSKFGCDKNSVDTNTKVWECKKPYKLSTKDVCVPPRRQELCLGNIDRIYDKNLLMIKEHILAIAIYESRILKRKYKNKDDKEVCKIINKTFADIRDIIGGTDYWNDLSNRKLVGKINTNSNYVHRNKENDKLFRDAWWKVIKKDVWNVISWVFKDKTVCKEDDIENIPQFFRWFSEWGDDYCQDKTKMIETLKVECKEKPCEDDNCKSKCNSYKEWISKKKEEYNKQAKQYQEYQKGNNYKMYSEFKSIKPEVYLKKYSKKCSNLNF

SEQ ID NO: 34 - protein, 114 aa, Artificial Sequence
  Fragment of MSP1
LEKHVCINTRDIPANAGCFRYDNGNEEWRCLLGYKKNNNTCIEDSNPTCGNNNGGCDPTAGCQTAENRENSKKIICTCKEPTPNAYYDGVFCSSSSFMGLSILLIITLIVFNIF

SEQ ID NO: 35 - protein, 641 aa, Artificial Sequence
  GBSSI-MSP1
ALDIVMVAAEVAPWSKTGGLGDVTGGLPIELVKRGHRVMTIAPRYDQYADAWDTSVVVDIMGEKVRYFHSIKKGVHRVWIDHPWFLAKVWGKTGSKLYGPRSGADYLDNHKRFALFCKAAIEAARVLPFGPGEDCVFVANDWHSALVPVLLKDEYQPKGQFTKAKSVLAIHNIAFQGRMWEEAFKDTKLPQAAFDKLAFSDGYAKVYTEATPMEEDEKPPLTGKTYKKINWLKGGIIAADKLVTVSPNYATEIAADAAGGVELDTVIRAKGIEGIVNGMDIEEWNPKTDKFLSVPYDQNSVYAGKAAAKEALQAELGLPVDPTAPLFAFIGRLEEQKGVDIILAALPKILATPKVQIAILGTGKAAYEKLVNAIGTKYKGRAKGVVKFSAPLAHMLTAGADFMLVPSRFEPCGLIQLHAMHYGTVPVVASTGGLVDTVKEGVTGFHMGALNPDKLDEADADALAATVRRASEVFAGGRYPEMVANCISQDLSWSKPAQKWEGLLEEVVYGKGGVATAKKEEIKVPVALEKHVCINTRDIPANAGCFRYDNGNEEWRCLLGYKKNNNTCIEDSNPTCGNNNGGCDPTAGCQTAENRENSKKIICTCKEPTPNAYYDGVFCSSSSFMGLSILLIITLIVFNIF

SEQ ID NO: 36 - protein, 106 aa, Artificial Sequence
  Fragment of AMA1
LEEFEEQFPCDIYKNKINEEIKVLNKNISNGNNSIEFPRIFISTDKNSLNCPCEPTQLTESSCNFYVCNCVEKRQYIAENNDVEIKEELEVHMKAHQTREVIVIII

SEQ ID NO: 37 - protein, 633 aa, Artificial Sequence
  GBSSI-AMA1
ALDIVMVAAEVAPWSKTGGLGDVTGGLPIELVKRGHRVMTIAPRYDQYADAWDTSVVVDIMGEKVRYFHSIKKGVHRVWIDHPWFLAKVWGKTGSKLYGPRSGADYLDNHKRFALFCKAAIEAARVLPFGPGEDCVFVANDWHSALVPVLLKDEYQPKGQFTKAKSVLAIHNIAFQGRMWEEAFKDTKLPQAAFDKLAFSDGYAKVYTEATPMEEDEKPPLTGKTYKKINWLKGGIIAADKLVTVSPNYATEIAADAAGGVELDTVIRAKGIEGIVNGMDIEEWNPKTDKFLSVPYDQNSVYAGKAAAKEALQAELGLPVDPTAPLFAFIGRLEEQKGVDIILAALPKILATPKVQIAILGTGKAAYEKLVNAIGTKYKGRAKGVVKFSAPLAHMLTAGADFMLVPSRFEPCGLIQLHAMHYGTVPVVASTGGLVDTVKEGVTGFHMGALNPDKLDEADADALAATVRRASEVFAGGRYPEMVANCISQDLSWSKPAQKWEGLLEEVVYGKGGVATAKKEEIKVPVALEEFEEQFPCDIYKNKINEEIKVLNKNISNGNNSIEFPRIFISTDKNSLNCPCEPTQLTESSCNFYVCNCVEKRQYIAENNDVEIKEELEVHMKAHQTREVIVIII

SEQ ID NO: 38 - protein, 57 aa, Artificial Sequence
  Peptide signal
MAVASTSRPSSARPIVINAASFGVKKTANQLLRELARGSARKSTSRSAVTGATGATC

SEQ ID NO: 39 - DNA, 351 bp, Artificial Sequence
  Fragment MSP1
ctcgagaagcacgtgtgcatcaacacgcgcgacattccggcgaacgctggctgtttccgctacgacaacggcaacgaggagtggcgctgcctgctgggctacaagaagaacaacaatacttgcatcgaggatagcaaccccacctgcggcaacaacaacggcggttgcgatcccaccgctggttgccagaccgccgagaaccgcgagaacagcaagaagatcatctgcacctgcaaggagccgaccccgaacgcctactacgacggcgtgttctgcagcagcagcagcttcatgggcctgagcatcctcctcatcattaccctgatcgtgttcaacatcttctaaggatcc

SEQ ID NO: 40 - DNA, 327 bp, Artificial Sequence
  Fragment AMA1
ctcgaggagttcgaggagcagttcccctgcgacatctacaagaacaagatcaacgaggagatcaaggtgctgaacaagaacatctccaacggcaacaacagcatcgagttcccccgcatcttcatcagcaccgacaagaacagcctgaactgcccctgcgagcccacccagctgaccgagagcagctgcaacttctacgtgtgcaactgcgtggagaagcgccagtacatcgccgagaacaacgacgtggagatcaaggaggagctggaggtgcacatgaaggcccaccagacccgcgaggtgatcgtgatcatcatctaaggattc

SEQ ID NO: 41 - DNA, 7239 bp, Artificial Sequence
  Plasmid pKB101
ctagagtcgacagccatatcgccgccgctttggccacctccaaacagccccctccccgcaaagccgcacatgctgccggcggggcgtcacacaccagacagaccagaccagcccgacattcaacacacacatggtctcatgcggtctgatggctttcctaagccaaccaggccggctcccagtgcagtgacgtgggcgtgacaggccgggtgctcccagccgcgtgccaattgccaaccccaccctacgcgaaggcattacgcgcctcaccgtgcattgctcctgctacagccccttgcaacaccgccgacctcgggaaggtggagttctcagcgcggtggccgcttgccccggccggcagctccgcagggcacacgtcacgcgaagggccgcgacggttcgagaaccgacttgagggcgccaaacgagcccgagccgccgttgcgccaggcgaaaccagaaccgtagattaatgcacttgagctattcattggagcgatctgccggggacagcgggtctggcgtgcgcgcgattggagatcgcaaattacatatgtctgcgtgacggcggggagctcgctgaggcttgacatgattggtgcgtatgtttgtatgaagctacaggactgatttggcgggctatgagggcgggggaagctctggaagggccgcgatggggcgcgcggcgtccagaaggcgccatacggcccgctggcggcacccatccggtataaaagcccgcgaccccgaacggtgacctccactttcagcgacaaacgagcacttatacatacgcgactattctgccgctatacataaccactcagctagcgatcccgggcgcgccagaaggagcgcagccaaaccaggatgatgtttgatggggtatttgagcacttgcaacccttatccggaagccccctggcccacaaaggctaggcgccaatgcaagcagttcgcatgcagcccctggagcggtgccctcctgataaaccggccagggggcctatgttctttacttttttacaagagaagtcactcaacatcttaaaatggctgttgcctctaccagccgccccagcagcgcgcgtcctatcgtcatcaatgccgcgtcgttcggtgtcaagaagaccgcgaaccagctgctgcgtgagcttgctcgtggctccgcacgcaagtccacctcgcgctcggctgttactggtgccactggtgccacttgcgcgctggacatcgtgatggttgctgctgaggtcgccccttggtccaagacgggcggtctgggcgatgtgactggtggcctgcctattgagctggtcaagcgcggccaccgcgtcatgaccattgcccctcggtaacgaccaaggaattcatgcacaaacgtgggctagcatcctctggcctggcgccatcatgtagcagtcttgagctgacatcaaacattggtaatcctcttgcaaacgtgaaacgatattgctgcactaccaagtaattcaggcgcgctgcgtaagcgactagtactgcacagatgttgcgcgtctcgcggccttaacgcctacgcatagtccgtttcgctccgttcgccaagctggggggctgtctctgtcacggcctgtgacttcgctatacgctgactatccacgtcgttgctgaaagcgctgtagagcctgggtatgtcgatcactgatggcgtttcatacctgcctccgcttgtttcacgcagctacgaccagtacgctgacgcctgggacacctcggtggtcgtggacatcatgggcgagaaggtccgctacttccactccatcaagaagggcgtgcaccgcgtgtggattgaccacccctggttcctggccaaggtctggggcaagaccggctccaagctgtacggcccccgctccggcgctgactacctggtgcgtggaggggcgctttgcatgacagtgctggcccatcatttgctcagccttgtctgcgctccgttgtatcatggcatgatgtgggtcggccctgcgtgtctctgaacctcactgcccccccttccatccactccccacaggacaaccacaagcgcttcgccctgttctgcaaggccgctattgaggctgcccgcgtgctgcccttcggccccggcgaggactgcgtcttcgtggccaacgactggcactccgccctggtgcccgtcctgctgaaggtgcgccgcatgaccgagcgccccatgtcagggcagcagcaatgctcccctcacctgcctgacagctggcttgctgaccagttcagcttgctgcccatcatggggttggcgcgacacgcctgtcctttctgcctccgcttgcgcatatgctgacctctacgctcgctgtcactccctcacctcgttactgctgcaggacgagtaccagcccaagggccagttcaccaaggccaagtcggtgctggctatccacaacatcgccttccagggccgcatgtgggaggaggctttcaaggacacgaagctgccccaggccgcctttgacaagctggccttctcggacggctatgccaaggtgcgcacagggtcttgcggaggtgctgctgcatgattgggctgctgacctaagggtgtgtctggcgctgtggtctctgacggactgggctgacctgcggaggcggcagcagaggtgggcctgtcagctgctgaccacggaaacacgctacgcctgcgcacgatgttctgcattctgttgtctgacggccacatcctcctgtctgcaagcaggtttacactgaggccacccccatggaggaggacgagaagcccccgctgacgggaaagacctacaagaagatcaactggctgaagggtggcattatcgccgccgacaagctggtgactgtgtcgcccaactacgcgaccgagatcgctgccgatgccgccggcggtgtggagctggacaccgtcatccggttgagaacccggaagtgtaaggagctgcaggacttgatgtggtgtcaatgaagggcggaacaacctgaagcggaaggctgttcttgactggttaaaagggctatgcttttcactacacagaacgaagcggctggcgcccaactgaccagcaccctgatcgctgactcgtcttgtccctctctcttgtgctgtcctctcctcccgcagcgccaagggcattgagggcattgtgaacggcatggacattgaggagtggaaccccaagaccgacaagttcctgtctgtgccctacgaccagaacagcgtctacgccggcaaggccgccgccaaggaggccctgcaggccgagctgggcctgcctgtggaccccaccgcccccctgttcgccttcatcggccgcctggaggagcagaagggtgtggacatcatcctggccgccctgcccaagatcctggccacccccaaggtgcagatcgccatcctgggtaccggcaaggccgcctacgagaagctggtgaacgccatcggcaccaagtacaagggccgcgccaagggcgtggtcaagttctcggcgcccctggcgcacatgctcaccgccggcgccgacttcatgctggtgccctcgcgcttcgagccctgcggcctgatccagctgcacgccatgcactacggtaccgtgcccgtggtagcctccaccggcggcctggtcgacaccgtcaaggagggcgtcaccggcttccacagtgagcggggcttcgcgtggtggtcgtgaacttgtgccctgaagtggtcgttgcgggggcgccttgacgggctcctgcagctgctaccctctgctgcgccggccaggaacccaccggtatcggaagcaccgtggcgcatgctgacgcctcccttttccccctgttccctgatcgcagtgggcgccctgaaccccgacaagctggacgaggctgacgccgacgccctggccgccaccgtgcgccgtgccagcgaggtgtttgcgggcggccgctaccccgagatggtggccaactgcatcagccaggacctgtcctggtccaagcccgcccagaagtgggagggcctgctggaggaggtggtgtacggcaagggcggcgtggccaccgccaagaaggaggagatcaaggtgcccgttgccctcgagggatccccgctccgtgtaaatggaggcgctcgttgatctgagccttgccccctgacgaacggcggtggatggaagatactgctctcaagtgctgaagcggtagcttagctccccgtttcgtgctgatcagtctttttcaacacgtaaaaagcggaggagttttgcaattttgttggttgtaacgatcctccgttgattttggcctctttctccatgggcgggctgggcgtatttgaagcgggtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctccaccgcggtggcggccgct